(19) United States
(12) Patent Application Publication
(10) Pub. No.: US 2024/0043486 A1
(43) Pub. Date: Feb. 8, 2024

(54) MANUFACTURE OF GRANULOCYTE MACROPHAGE-COLONY STIMULATING FACTOR

(71) Applicant: Partner Therapeutics, Inc., Lexington, MA (US)

(72) Inventors: Greg MILLER, Lexington, MA (US); Shawn LILLIE, Lexington, MA (US); Jason IRELAND, Lexington, MA (US)

(21) Appl. No.: 18/265,508

(22) PCT Filed: Dec. 7, 2021
(86) PCT No.: PCT/US21/62168

§ 371 Date: Jun. 6, 2023

Related U.S. Application Data
(60) Provisional application No. 63/271,444, filed on Oct. 25, 2021.
(60) Provisional application No. 63/122,593, filed on Dec. 8, 2020.

(51) Int. Cl. C07K 14/535; C12P 21/02

(52) U.S. Cl. CPC C07K 14/535; C12P 21/02; C12N 2500/10; A61K 38/00

(57) ABSTRACT

The present disclosure relates to a manufacturing process of sargramostim, which results in improved yield efficiency and output.

### PRIORITY

The present application claims priority to and benefit from U.S. Provisional Patent Application No. 63/122,593, filed Dec. 8, 2020 and U.S. Provisional Patent Application No. 63/271,444, filed Oct. 25, 2021, the entirety of each which is incorporated by reference herein.

### FIELD OF THE INVENTION

The present invention relates generally to methods related to improving and increasing yield of granulocyte-macrophage colony-stimulating factor (GM-CSF).

### DESCRIPTION OF THE TEXT FILE SUBMITTED ELECTRONICALLY

This application contains a Sequence Listing in ASCII format submitted electronically herewith via EFS-Web. Said ASCII copy, created on Dec. 6, 2021, is named PNR-004PC_SequenceListing_ST25.txt and is 4,096 bytes in size. The Sequence Listing is incorporated herein by reference in its entirety.

### BACKGROUND

Colony Stimulating Factor, CSF, refers to a family of four glycoproteins that control and coordinate cell production by widely scattered deposits of marrow cells. These include: Granulocyte-Macrophage CSF (GM-CSF), Granulocyte colony CSF (G-CSF), Macrophage colony CSF (M-CSF) and multipotential colony-stimulating factor (IL-3). These lymphokines can induce progenitor cells found in the bone marrow to differentiate into specific types of mature blood cells. The particular type of mature blood cell that results from a progenitor cell depends upon the type of CSF present. See Metcalf D. Cancer Immunol Res. 2013, 1(6): 351-356.

GM-CSF is a hematological growth factor that regulates the production, migration, proliferation, differentiation and function of hematopoietic cells. In response to inflammatory stimuli, GM-CSF is released by various cell types including T lymphocytes, macrophages, fibroblasts and endothelial cells. GM-CSF then activates and enhances the production and survival of neutrophils, eosinophils, and macrophages. Native GM-CSF is usually produced near the site of action where it modulates in vitro proliferation, differentiation, and survival of hematopoietic progenitor cells, but is present in circulating blood in only picomolar concentrations (10⁻¹⁰ to 10⁻¹² M). See Alexander W S. Int Rev Immunol. 1998, 16:651-682; Gasson J C. Blood. 1991, 77:1131-1145; Shannon M F et al. Crit Rev Immunol. 1997, 17:301-323, Barreda D R et al. Dev Comp Immunol. 2004, 28:509-554 and Metcalf D. Immunol Cell Biology. 1987, 65:35-43.

Human GM-CSF (hGM-CSF) is synthesized as a 144 amino acid residue precursor protein with a 17 amino acid signal peptide. This precursor protein is processed to yield a 127 amino acid mature protein with a predicted molecular mass of 14.4 kDa. It has two disulfide linkages that migrates as a broad band of 15-30 kDa due to glycosylation and sialylation. The glycosylation patterns of GM-CSF have been observed to influence its activity, receptor binding, immunogenicity, and half-life. See Lee F. et al. Proc Natl Acad Sci USA Biochem. 1985. 82: 360-4364; Miyatake S. et al. EMBO J. 1985. 4: 2561-2568. Cebon J et al. J Biol. Chem. 1991. 265, 4483-4491; Zhang Q et al. Proc. Natl. Acad. Sci. 2014. 2885-2890.

Recombinant human granulocyte-macrophage colony-stimulating factor (rhu GM-CSF) has been approved by the FDA for the treatment of neutropenia, blood dyscrasias and malignancies like leukemia in combination with chemotherapies. In the clinic, GM-CSF used for treatment of neutropenia and aplastic anemia following chemotherapy greatly reduces the risk of infection associated with bone marrow transplantation. Its utility in myeloid leukemia treatment and as a vaccine adjuvant is also well established. See Dorr R T. Clin Therapeutics. 1993. 15(1):19-29; Armitage J O. Blood 1998, 92:4491-4508; Kovacic J C et al. J Mol Cell Cardiol. 2007, 42:19-33; Jacobs P P et al. Microbial Cell Factories 2010, 9:93.

Although there are five classes of heterologous protein production platforms, including bacteria, yeasts, plants, insect cells, and mammalian cells, more than 50% of currently marketed biopharmaceuticals are produced in mammalian cell lines. This is in part due to the inability of the remaining four classes to modify glycoproteins with human-like oligosaccharides. This is of importance as protein-bound glycans influence circulation half-life, tissue distribution, biological activity and immunogenicity. The GM-CSF expression system influences the pharmacokinetics properties, biological activity and clinical toxicity of GM-CSF. In the clinic, GM-CSF has been produced in Chinese hamster ovary cells (CHO-GM, regramostim), Escherichia coli (E. coli-GM, molgramostim), or yeast (Yeast-GM, sargramostim). See Dorr R T. Clin Therapeutics. 1993. 15(1):19-29; Walsh G. Nat Biotechnol. 2006, 24:769-776; Jacobs P P et al. Nat Protoc. 2009, 4:58-70; Jacobs P P et al. Microbial Cell Factories 2010, 9:93; Walsh G. Nat Biotechnol. 2018, 36(12): 1136-1145.

In addition to water and oxygen, the basic nutritional requirements for all microorganisms include carbon, nitrogen, vitamins and mineral elements. The mineral requirements in yeast vary depending upon the specific stain and culture growth conditions. In general, yeast have two types of mineral requirements; macro elements, or those required in larger amount and micro elements, or those required in trace amounts. The micro or trace elements include iron, copper, zinc, manganese, molybdenum, cobalt, boron and others. These trace elements are essential in the growth of yeast and play an important role in cellular metabolism, primarily due to their requirements as cofactors for a large number of enzymes. In the sargramostim cell expansion steps of the manufacturing process (shake flask, seed fermentation), the mineral requirements of the host organism are met by addition of a trace elements solution to the media. However, in the production fermentation trace elements are not added, but rather a blend of two complex protein hydrolysates are used to satisfy all the mineral requirements (Bacto-Peptone, Yeast Extract).

Bacto-Peptone and Yeast Extract are utilized in the sargramostim manufacturing process as a complex organic nitrogen, inorganic nitrogen, vitamins, trace elements and free amino acids source for the yeast culture during the production fermentation, thereby promoting cell proliferation and expression and secretion of sargramostim. The heterogeneous nature of these materials and associated lot-to-lot variation has been shown to significantly affect yeast culture performance, productivity and product quality. As a result, the rate of growth and productivity may be strongly affected by unknown mineral variations provided to the culture through the complex media.

There remains a need for reducing the variation in the micronutrients during the manufacturing process of rhu GM-CSF to improve yield consistency and efficiency.

### SUMMARY OF THE INVENTION

Accordingly, the present invention, in part, relates to copper, an essential micro-element in yeast, as a principle limiting component in the media affecting productivity. For instance, the disclosure demonstrates, inter alia, that copper (Cu) is the limiting trace element in the Bacto Peptone and Yeast Extract. Supplementation of additional copper to the media improved poor producing lots, resulting in a significant yield increase.

In aspects, there is provided a method for production of a recombinant protein, comprising adding a trace element, copper, to a culture medium comprising a host cell, such as yeast. The host cell comprises a nucleic acid molecule encoding the recombinant protein, e.g. rhu GM-CSF, and is capable of producing this protein during fermentation and capable of producing the recombinant protein during fermentation, and this trace element is exogenously added to the culture medium to supplement an amount of trace element in the culture medium.

In embodiments, there is also provided methods for production using nucleic acid molecules encoding the present recombinant human GM-CSF (e.g. a codon-optimized sequence). In embodiments, there is also provided methods for production using a non-human host cell expressing the nucleic acid molecule encoding the present recombinant human GM-CSF (e.g. a yeast cell, e.g. a non-methylotrophic yeast cell, e.g. a Saccharomyces cerevisiae). In embodiments, there is also provided a pharmaceutical composition comprising the present recombinant human GM-CSF and a pharmaceutically acceptable excipient or carrier, produced by the present methods for production.

In aspects, there is provided a method of treating a patient or subject who is undertaking or has undertaken a cancer therapy, or who is undertaking or has undertaken a bone marrow transplant, and/or who had been acutely exposed to myelosuppressive doses of radiation; the method comprising administering to the patient a therapeutically effective amount of the pharmaceutical compositions, produced by the present methods for production, described herein.

In aspects, there is provided a method of treating a viral infection, e.g. without limitation an infection with a coronavirus, e.g. without limitation severe acute respiratory syndrome coronavirus 2 (SARS-CoV-2), comprising administering an effective amount of the pharmaceutical compositions, produced by the present methods for production, described herein, or a method for treating or preventing a viral infection in a subject in need thereof, by providing plasma from a donor subject who has recovered from the viral infection, e.g. without limitation an infection with a coronavirus, e.g. without limitation SARS-CoV-2, the plasma comprising IgG, IgM and/or IgA antibodies directed against the virus causing the infection and the donor subject having been treated with the recombinant human GM-CSF protein, produced by the present methods for production, described herein to stimulate production of the antibodies; and administering the plasma to the subject in need thereof.

In aspects, there is provided a method of method of making a recombinant producing a composition comprising a recombinant human GM-CSF comprising: (a) adding an exogenous trace element, copper, to a culture medium comprising a host cell such as yeast, and this trace element is exogenously added to the culture medium to supplement an amount of trace element in the culture medium to achieve a target concentration range; (b) transfecting the yeast cell with a nucleic acid encoding a recombinant human GM-CSF, comprising an amino acid sequence at least about 97% identical with, or at least about 98% identical with, at least about 99% identical with, or having the amino acid sequence of SEQ ID NO: 1 and/or SEQ ID NO: 2 and (c) the host cell capable of producing this protein during fermentation with increased efficacy and consistency.

In aspects, the present invention relates to a method for improving the production of a physiologically active substance, such as recombinant human GM-CSF, comprising adding exogenous copper to a culture medium for the production of a physiologically active substance obtainable by culturing an animal cell or cell line which is capable of producing the physiologically active substance in the culture medium.

More specifically, the present invention, in embodiments, relates to a method for producing a physiologically active substance, comprising culturing an animal cell (such as yeast cells) or cell line (such as CHO cells) which is capable of producing a physiologically active substance in a culture medium containing exogenous copper to produce the physiologically active substance; and isolating the physiologically active substance from the culture medium.

### BRIEF DESCRIPTION OF DRAWINGS

The patent or application file contains at least one drawing executed in color. Copies of this patent or patent application publication with color drawings will be provided by the Office upon request and payment of the necessary fee

FIG. 1A illustrates the effect of the various trace elements on the quantity of dissolved oxygen following addition to the yeast cell culture. Dissolved oxygen profiles are shown, in which a comparison of trace elements was screened individually. The bottom curve is “Copper”.

FIG. 1B illustrates the effect of the addition of exogenous copper on the quantity of dissolved oxygen following addition to the yeast cell culture as compared to the commercial scale-down process. Dissolved oxygen profiles are shown, in which a comparison of simultaneous fermentations is shown: commercial scale-down process and copper supplemented. At 15.0 hours, the top curve is “Commercial Scale Down Process” and the bottom curve is “Copper Supplemented”.

FIG. 2A illustrates the effect of the various trace elements on the wet cell weight of yeast following addition to the yeast cell culture. A wet cell weight profile is shown, in which a comparison of trace elements screened individually was made. At time=20 hours, the top curve is “Copper,” followed by “Zinc”, “Molybdate,” “Manganese,” “Iron,” and “Boron,” from top to bottom.

FIG. 2B illustrates the effect of the addition of exogenous copper on wet cell weight of yeast following addition to the yeast cell culture as compared to the commercial scale-down process. A bar graph of wet cell weight is shown, with comparison of simultaneous fermentations: commercial scale-down process and copper supplemented demonstrated.

FIG. 3 illustrates the titers of recombinant human GM-CSF obtained during simultaneous fermentation with or without the addition of exogenous copper. A bar graph of various titers, with comparison of simultaneous fermentations: commercial scale-down process and copper supplemented.

FIG. 4 illustrates the results from SDS-PAGE-Silver Stain (T-0002) assay to evaluate impurities for the CuSO4 batch at BDS (CuSO4 PV) compared to commercial BDS batches 6-8. Each gel contains a reference standard, molecular weight marker, and reduced and non-reduced samples. Sample identity is as follows: BDS 6: Ref Std. reduced (lane 2), BDS 6 reduced (lane 4), Ref. Std non-reduced (lane 7) and BDS 6 non-reduced (lane 9). BDS 7: Ref Std. reduced (lane 2), BDS 7 reduced (lane 5), Ref. Std non-reduced (lane 7) and BDS 7 non-reduced (lane 10). BDS 8: Ref Std. reduced (lane 2), BDS 8 reduced (lane 3), Ref. Std non-reduced (lane 7) and BDS 8 non-reduced (lane 8), CuSO4 PV: Ref Std. reduced (lane 2), CuSO4 PV reduced (lane 3), Ref Std. non-reduced (lane 7), CuSO4 PV non-reduced (lane 8).

FIG. 5 illustrates the results from densitometry testing (T-0013) to evaluate the level of protein purity of the sargramostim for the CuSO4 batch at BDS (CuSO4 PV) compared to commercial BDS batches 6-8. Each gel contains a reference standard lane (lane 4), thermo molecular weight marker (lane 2) and commercial BDS or PV sample (lane 6).

FIG. 6 illustrates the results from isoelectric focusing (T-0114) which was used to determine the identity of the sargramostim for the CuSO4 batch at BDS (CuSO4 PV) compared to commercial BDS batches 6-8. Each gel contains a GE healthcare pl marker (lane 2), reference standard (lane 4) and commercial BDS or PV sample (lane 6).

FIG. 7 illustrates the results of ELISA showing the residual process components (RPC) removal throughout the purification process in the CuSO4 PV batch (CuSO4 PV) versus all historic batches. The dotted line depicts the average of all historical commercial data, the solid line depicts CuSO4 batch at BDS (CuSO4 PV). Commercial BDS batches 6-8 are shown at the BDS level only. The results of all historic commercial batches, CuSO4 PV and BDS 6-8 are very similar and overlap.

FIG. 8 illustrates RP-HPLC chromatographic peak separation showing that C-term inal analysis that was performed utilizing a tryptic peptide map (TCPK-Trypsin). Peak A (Amino Acids 86-107), Peak B (Amino Acids 108-111), and Peak C (Amino Acids 112-127) for each of the CuSO4 PV and commercial BDS 6-8.

FIG. 9 illustrates the low pH Glu-C peptide map which depict the disulfide bridge pairing. The chromatograms show peaks 11 and 12 which contain the disulfide peptide fragments which are confirmed by mass spec analysis. The figure shows CuSO4 batch at BDS (CuSO4 PV) as well as the commercial BDS batches 6-8.

FIG. 10 illustrates the low pH Glu C peptide map chromatogram (78.5-82.5 min) containing the peptides G3-4 and deamidated fragments. The results show the total percentage of N-linked glycosylation (site occupancy) at position 27. The figure shows CuSO4 batch at BDS (CuSO4 PV) as well as the commercial BDS batches 6-8.

FIG. 11 illustrates the Glu C peptide map without α-mannosidase chromatograms containing the glycosylated G1 peptides, non-glycosylated Ala 3 and non-glycosylated Ala 1 peptide fragments. The total O-linked glycosylation chain size (site occupancy) was determined by the total area of the O-linked glycoform peaks compared to the unmodified area expressed as a percent. The figure shows CuSO4 batch at BDS (CuSO4 PV) as well as the commercial BDS batches 6-8.

FIG. 12 illustrates the neutral pH Glu C peptide map chromatogram containing the G9 and oxidized fragment. Oxidation at methionine 79 was determined by mass spectrometry. The figure shows CuSO4 batch at BDS (CuSO4 PV) as well as the commercial BDS batches 6-8.

FIG. 13 illustrates the blank subtracted emission fluorescence spectra from 305 nm-405 nm from Excitation=295 nm. The spectral graphs show the comparability in the thermal stability of the protein structures when measured between 10°−90° C. The figure shows graphs for CuSO4 batch at BDS (CuSO4 PV) as well as the commercial BDS batches 6-8. Curves indicate measurements from about 10° C.-18° C. (in purple curves) starting at the top of FIG. 13 to about 20° C.-32° C. (in blue curves) to about 34° C.-46° C. (in green curves) to about 48° C.-52° C. (in yellow curves) to about 54° C.-58° C. (in orange curves) to about 60° C.-80° C. (in red curves) to about 82° C.-90° C. (in brown curves) ending at the bottom of FIG. 13.

FIG. 14 illustrates the center of spectral mass of 305-405 nm emission spectra to show the comparability in protein structure in solution between the lots. The figure shows CuSO4 batch at BDS (CuSO4 PV) as well as the commercial BDS batches 6-8.

FIG. 15 illustrates circular dichroism (CD) spectral comparison (5-10° C. and 90° C.) graphs. The CD scans and thermal unfolding data (Tₘ and Tₒₙₛₑₜ) show the comparability amongst the all the four BDS (CuSO4 PV and commercial BDS 6-8) lots tested. The red line illustrates absorption at 90° C. and the blue line illustrates absorption at 10° C.

FIG. 16 shows the intact or full MALDI-MS mass spectra analysis from 12 to 19 KDa. The graphs illustrate the observed spectral masses for all four BDS (CuSO4 PV and commercial BDS 6-8) lots tested. All the MALDI-MS imaging was done at the Fred Hutchinson Cancer Research Center Proteomic Facility on an Applied Biosystems 4800 MALDI-TOF/TOF. The samples were diluted 10-fold with sinnapinic acid, spotted on a MALDI plate, and MS were acquired for 15 minutes per sample from 2 to 19 KDa.

FIG. 17 shows the MALDI-MS mass spectra analysis for sargramostim from 14 to 19 KDa. The graphs illustrate the observed spectral masses to for all four BDS (CuSO4 PV and commercial BDS 6-8) lots tested.

FIG. 18 shows the MALDI-MS mass spectra analysis for sargramostim from 16 to 19 KDa. The graphs illustrate the observed spectral masses to for all four BDS (CuSO4 PV and commercial BDS 6-8) lots tested.

### DETAILED DESCRIPTION

The present invention is based, in part, on the discovery that the exogenous addition of a single micronutrient, copper (Cu) during the manufacturing causes an increase in yield of recombinant human GM-CSF (rhu GM-CSF). Further, the present invention is based on the discovery that this increase in manufacturing efficiency had no impact on the quality of the rhu GM-CSF produced.

The present invention, in embodiments, provides a method for improving the production of a physiologically active substance, such as rhu GM-CSF, by adding exogenous copper to a culture medium for use in the production of the physiologically active substance by a cultured animal cell (such as yeast cells) or cell line (such as CHO cells).

### Methods of Making

In embodiments provided herein are methods for achieving consistent and efficient production of a recombinant glycoprotein, such as rhu GM-CSF, comprising increasing the concentration of copper in a cell culture to achieve a target concentration range, wherein the cell culture comprises host cells producing the recombinant glycoprotein of interest.

In embodiments provided herein are methods for improving a cell culture medium for the production of a recombinant rhu GM-CSF comprising (i) determining the amount of copper in a cell culture medium or a component used to produce a cell culture medium, and (ii) adjusting the concentration of copper in the cell culture medium to achieve an amount of copper within a predetermined target range, wherein the target range is sufficient to produce the recombinant glycoprotein of interest with increased consistency and yield.

In embodiments provided herein are methods for improving the production of a physiologically active recombinant glycoprotein such as rhu GM-CSF comprising (i) measuring the amount of copper in a cell culture of yeast and (ii) if the amount of copper is below a target range, supplementing the yeast cell culture with copper to achieve an amount of copper within the target range.

In aspects, there is provided a method of method of making a recombinant producing a composition comprising a recombinant human GM-CSF comprising: (a) obtaining a yeast cell transfected with a nucleic acid encoding a recombinant human GM-CSF, comprising an amino acid sequence having at least about 90%, at least about 93%, at least about 95%, at least about 97%, at least about 98%, or at least about 99% identity with SEQ ID NO: 1 or SEQ ID NO: 2, or an extract thereof; (b) purifying the GM-CSF from the transfected yeast cell using one or more HPLC columns, wherein the purification is in the absence of an organic solvent; and (c) collecting the purified GM-CSF, the purified GM-CSF being substantially free of hyperglycosylated, e.g. hypermannosylated GM-CSF forms.

In embodiments, the yeast is S. cerevisiae.

In embodiments, the method further comprises formulating the purified GM-CSF for injection, e.g. subcutaneous or intravenous injection.

### Culture Medium

In embodiments, the culture medium of the present invention is not particularly limited, so long as it can sustain the survival and growth of animal cells (such as yeast cells) or cell lines (such as CHO cells). Examples include media containing a carbon source that can be assimilated by animal cells, a nitrogen source that can be digested thereby, vitamins and/or mineral elements. In embodiments, the culture medium comprises bacto-peptone and/or yeast extract.

In embodiments, the mineral elements of the present invention comprise macro and micro elements. Such macro elements include carbon, hydrogen, oxygen and nitrogen. Examples of micro elements include copper, iron, zinc, manganese, molybdenum, cobalt, boron and the like.

In embodiments, the culture medium is supplemented with additional exogenous trace mineral elements such as copper. Such supplementation of the cell culture medium as in the present invention can control manufacturing efficiency and productivity.

Without wishing to be bound by theory, the nutritional requirements of yeast that can influence rate of growth and survival (Duc C et al., PLOS One, 12(9): 1-22; Broach J R Genetics. 192(1):73-105, 2012; Gadd G M, FEMS Microbial Lett. 79:197-203, 1992).

### Copper

In embodiments, copper can be added to the cell culture medium in the form of copper or cupric sulfate. The amount of copper is added to the cell culture medium in an amount of about 0.5 μM to about 100 μM, optionally being about 0.5 μM to about 80 μM, or optionally being about 1 μM to about 20 μM depending on the particular culture medium.

In embodiments, copper can be added to the cell culture in the form of copper (cupric) sulfate or copper oxide or copper chloride or copper iodide or copper sulfide or copper acetylide or copper bromide or copper fluoride or copper hydroxide or copper hydride or copper nitrate or copper phosphide or copper acetate or copper carbonate or copper chlorate or copper phosphate.

Accordingly, in embodiments, this information may inform a skilled artisan with regard to acceptable variations in the copper salts.

### Fermentation

In embodiments, the present invention provides for methods that involve fermentation to yield a protein product.

In various embodiments, the manufacturing of the recombinant protein, e.g. the engineered rhu GM-CSF can be comprised of a series of ten or up to ten distinct unit operations. In embodiments, the recombinant protein, e.g. the sargramostim manufacturing fermentation process generates rhu GM-CSF for harvest and recovery. During the upstream manufacturing process, four major GM-CSF species, including a hyper-glycosylated isoform, N- and N-+0-glycosylated isoform, an O-glycosylated isoform and an non-glycosylated (˜15 kDa, peak 4) species are present in partially purified fermenter broth.

In various embodiments, the fermentation process has three stages: 1.5 L Shake Flask, 15 L Seed Fermentation and 100 L Production Fermentation. The 1.5 L Shake Flask step is a process that can expand the preliminary yeast culture from a Working Cell Bank vial to a volume and density sufficient to inoculate the 15 L Seed Fermentation process. The 15 L Seed Fermentation is a process that can further expand the culture to a volume and density sufficient to inoculate the 100 L Production Fermentation. The 100 L Production Fermentation is a fed-batch process that can increase the biomass and promotes the expression and secretion of the recombinant protein, e.g. the rhu GM-CSF into the fermentation medium for subsequent harvest and purification. In embodiments, at the end of the 100 L Production Fermentation process, fermentation cultures are combined for harvest by microfiltration and ultrafiltration.

### Isolation

In embodiments, the present invention provides for methods that involve isolation methods to yield a protein product. In some embodiments, the purification or isolation of the recombinant protein, e.g. engineered rhu GM-CSF is isolated or purified on the basis of such characteristics as solubility, size, charge, and specific binding affinity, e.g. by gel-filtration chromatography, ion-exchange chromatography, affinity chromatography, or high-pressure liquid chromatography.

In some embodiments, the purification or isolation of the recombinant protein, e.g. engineered rhu GM-CSF takes places in the downstream processing consists of three Reverse Phase-High Pressure Liquid Chromatography (RP-HPLC) operations, one low pressure cation exchange chromatography operation and a final filtration operation. In some embodiments, the purification or isolation step can include a C4 capture process, a C4 purification process and a C18 purification process.

### Compositions of GM-CSF

In an embodiment, the engineered rhu GM-CSF manufactured using the present invention of the addition of exogenous copper is the same as recombinant human GM-CSF (rhu GM-CSF), such as sargramostim (LEUKINE). Sargramostim is a biosynthetic, yeast-derived, recombinant human GM-CSF, having of a single 127 amino acid glycoprotein that differs from endogenous human GM-CSF by having a leucine instead of a arginine at position 23. Other natural and synthetic GM-CSFs, and derivatives thereof having the biological activity of natural human GM-CSF, may be equally useful in the practice of the invention.

Without wishing to be bound by theory, the degree of glycosylation of biosynthetic GM-CSFs appears to influence half-life, distribution, and elimination. (Lieschke and Burgess, N. Engl. J. Med. 327:28-35, 1992; Dorr, R. T., Clin. Ther. 15:19-29, 1993; Norgaard et al., Eur. J. Hematol. 50:32-36, 1993).

In an embodiment, there is provided a recombinant human GM-CSF protein, comprising an amino acid sequence having at least about 90%, or at least about 91%, or at least about 92%, or at least about 93%, or at least about 94%, or at least about 95%, or at least about 96%, or at least about 97%, or at least about 98%, or at least about 99% identity, or 100% identity with SEQ ID NO: 1 or SEQ ID NO: 2.

In embodiments, the GM-CSF is one of molgramostim, sargramostim, and regramostim.

Without wishing to be bound by theory, the core of hGM-CSF consists of four helices that pack at angles. Crystal structures and mutagenic analysis of recombinant human GM-CSF (Rozwarski D A et al., Proteins 26:304-13, 1996) showed that, in addition to apolar side chains in the protein core, 10 buried hydrogen bonding residues involve intramolecular hydrogen bonding to main chain atoms that were better conserved than residues hydrogen bonding to other side chain atoms; 24 solvation sites were observed at equivalent positions in the two molecules in the asymmetric unit, and the strongest among these was located in clefts between secondary structural elements. Two surface clusters of hydrophobic side chains are located near the expected receptor binding regions.

Further, in embodiments, one of ordinary skill can reference UniProtKB entry P04141 for structure information to inform the identity of variants.

The N-terminal helix of hGM-CSF governs high affinity binding to its receptor (Shanafelt A B et al., EMBO J 10:4105-12, 1991). Transduction of the biological effects of GM-CSF requires interaction with at least two cell surface receptor components, (one of which is shared with the cytokine IL-5). The above study identified receptor binding determinants in GM-CSF by locating unique receptor binding domains on a series of human-mouse hybrid GM-CSF cytokines. The interaction of GM-CSF with the shared subunit of their high affinity receptor complexes was governed by a very small part of the peptide chains. The presence of a few key residues in the N-terminal α-helix of was sufficient to confer specificity to the interaction.

In embodiments, the engineered GM-CSF used in the practice of the invention includes any pharmaceutically safe and effective GM-CSF, or any derivative thereof having the biological activity of GM-CSF.

In embodiments, the present rhu GM-CSF molecules comprise a plurality of molecular forms similar to sargramostim. In embodiments, the molecular forms are selected from non-glycosylated, 0-glycosylated, N-glycosylated and N+0 glycosylated forms. Further in embodiments, the recombinant human GM-CSF is substantially free of hyperglycosylated, e.g. hypermannosylated forms.

In embodiments, the present rhu GM-CSF comprises more than one species (e.g. glycoforms). In embodiments, none of the species have a molecular weight of greater than about 20 kDa.

### Functional Properties of the Recombinant GM-CSF

In embodiments, the present recombinant human (rhu) GM-CSF molecules manufactured with the addition of exogenous copper is functionally similar to wild type human GM-CSF and/or sargramostim made without the addition of exogenous copper (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold of the assayed functional parameter). In embodiments, the functional parameters of GM-CSF can be detected by assays known in the art, e.g., without limitation, proliferation assays using cells such as TF-1 cell lines, primary bone marrow cells, biochemical assays such as iLite™ GM-CSF (luciferase under the control of GM-CSF promoter), cell survival assays e.g. myeloid cell survival assay, cell differentiation assays and co-culture experiments.

In embodiments, the present rhu GM-CSF molecules manufactured with the addition of exogenous copper can bind and/or activate the granulocyte-macrophage colony stimulating factor receptor (GM-CSF-R-alpha or CSF2R). In embodiments, the present rhu GM-CSF molecules manufactured with the addition of exogenous copper can bind and/or activate the granulocyte-macrophage colony stimulating factor receptor (GM-CSF-R-alpha or CSF2R) at an affinity, efficacy, and/or bioactivity that is comparable to wild type human GM-CSF and/or sargramostim made without the addition of exogenous copper (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold). Assays for GM-CSF binding and activation are known in the art. Non-limiting examples of such assays include, for example, radioligand assays or non-radioligand assays (e.g. immunoprecipitation (IP), enzyme-linked immunosorbent assay (ELISA), western blot, fluorescence polarization (FP). Fluorescence resonance energy transfer (FRET), surface plasmon resonance (SPR), and radioimmunoassay (RIA). The binding kinetics also can be assessed by standard assays known in the art, such as by Biacore analysis. Whole cell ligand-binding assays, and cell-free assay systems using soluble GM-CSF receptor alpha (sGMRa) may also be used. Some other types of assays that may be used include, receptor-binding, or saturation binding, or competitive binding assays using radio-iodinated GM-CSF, as well as cell proliferation assays.

In embodiments, the present rhu GM-CSF molecules can be assayed using one or more cell-based activity bioassays, e.g. using a GM-CSF dependent human cell-line proliferation assay, e.g. using TF-1, M-07e, HU-3, M-MOK, MB-02, GM/SO, F-36P, GF-D8, ELF-153, AML-193, MUTZ-3, OCI-AMLS, OCI-AML6, OCI-AML1, SKNO-1, UCSD-AML1 and UT-7.

In embodiments, the potency of the present rhu GM-CSF molecules is measured using a bioassay employing TF-1 cells, a human erythroid leukemia cell line that proliferates in response to GM-CSF. The details of this assay are known in the art. For instance, a reference standard, control and test samples are serially diluted in triplicate in assay media and added to three separate 96-well plates. TF-1 cells in suspension are then added and the mixture is incubated at 37° C. for 69.5-72 hours. Following the addition of a fluorescent dye (e.g. ALAMARBLUE), the plates are incubated at 37° C. for 6.6-8 hours. TF-1 cell proliferation is then measured in a fluorescent microplate reader.

In embodiments, the GM-CSF-R-alpha at which binding and/or activation occurs is expressed on the surface of a cell. In embodiments, the cell is a hematopoietic progenitor cell. In embodiments, the hematopoietic progenitor cell is an immune cell. In embodiments, the hematopoietic progenitor cell is irradiated.

In embodiments, the immunogenicity of the present rhu GM-CSF molecules, with the present substitutions and/or deletions is comparable to wild type human GM-CSF and/or sargramostim (e.g. differ in one or more functional parameter by no more than about 50%, or by no more than about 40%, or by no more than about 30%, or by no more than about 20%, or by no more than about 10%, or by no more than about 5%, or no more than about 5-fold, or no more than about 4-fold, or no more than about 3-fold, or no more than about 2-fold). In embodiments, immunogenicity is assayed using methods known in the art. Non-limiting examples include detection of one or more anti-GM-CSF binding antibodies as assessed by, e.g. screening assays such as direct or indirect or bridging ELISA, electrochemiluminescence, bead-based chemiluminescence assays, radioimmunoprecipitation assay, surface plasma resonance and bio layer interferometry, as well as cell based luciferase reporter gene neutralizing antibody assay.

In embodiments, the cell recombinant human GM-CSF is soluble.

### Nucleic Acids and Host Cells

In embodiments, there is provided a nucleic acid molecule encoding the recombinant human GM-CSF described herein. In embodiments, the nucleic acid molecule has a codon-optimized sequence.

In embodiments, there is provided a non-human host cell expressing the nucleic acid molecule described herein. In embodiments, the host cell is a yeast cell.

In embodiments, the yeast cell is a non-methylotrophic yeast cell. In embodiments, the host cell is a Saccharomyces cerevisiae cell.

In embodiments, the host cell is a mammalian cell. In embodiments, the host cells are CHO (Chinese hamster ovary) cells, NSO (mouse myeloma) cells, BHK (baby hamster kidney) cells, Sp2/0 (mouse myeloma) cells, human retinal cells, HUVEC cells, HMVEC cells, COS-1 cells, COS-7 cells, HeLa cells, HepG-2 cells, HL-60 cells, IM-9 cells, Jurkat cells, MCF-7 cells or T98G cells, and the like.

### Pharmaceutical Compositions and Formulations

In embodiments, there is provided a pharmaceutical composition comprising a recombinant human GM-CSF described herein and a pharmaceutically acceptable excipient or carrier.

Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention, in embodiments, includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid

Where necessary, the present pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional therapeutic agents) of the present invention, in embodiments, may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional therapeutic agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In specific embodiments, the GM-CSF (and/or additional therapeutic agents) is administered via an intravenous route.

In one embodiment, the pharmaceutical compositions (and/or additional therapeutic agents) described herein are formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any pharmaceutical compositions (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Any inventive pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention, in embodiments, thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Pharmaceutically Acceptable Salts and Excipients

The compositions described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenyl butyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylam ine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

### Methods of Use

In an aspect, there is provided a method of treating a patient or subject who is undertaking or has undertaken a cancer therapy, or who is undertaking or has undertaken a bone marrow transplant, and/or who had been acutely exposed to myelosuppressive doses of radiation; the method comprising administering to the patient a therapeutically effective amount of the present recombinant human GM-CSF protein or a pharmaceutical composition thereof. In embodiments, the patient is treated by modulating clonal expansion, survival, differentiation and activation state of hematopoietic progenitor cells. In embodiments, the patient is treated by modulating a myelomonocytic cell lineage, by promoting the proliferation of megakaryocytic and erythroid progenitors. In embodiments, the patient is treated by modulating hematopoietic progenitor cells, by stimulating the survival, proliferation and activation of neutrophils, macrophages and/or dendritic cells. In embodiments, the patient is treated following bone marrow transplant by modulating hematopoietic progenitor cells, by stimulating the survival, proliferation and activation of neutrophils, macrophages and/or dendritic cells.

In an aspect, there is provided a therapeutic method comprising administering to a patient a therapeutically effective amount of the present recombinant human GM-CSF protein or a pharmaceutical composition thereof or contacting cells with an effective amount of the pharmaceutical composition described herein and administering therapeutically effective amount of the cells, wherein the therapy: accelerates neutrophil recovery and/or to reduce the incidence of infections following induction chemotherapy; mobilizes hematopoietic progenitor cells into peripheral blood for collection by leukapheresis and transplantation; accelerates of myeloid reconstitution following autologous or allogeneic bone marrow or peripheral blood progenitor cell transplantation; treats delayed neutrophil recovery or graft failure after autologous or allogeneic bone marrow transplantation; and/or treats hematopoietic syndrome of acute radiation syndrome (H-ARS).

In an aspect, there is provided a method for treating an infection with a virus, comprising: administering an effective amount of a composition comprising the present recombinant human GM-CSF protein or a pharmaceutical composition comprising the same to a patient in need thereof.

In embodiments, the viral infection is an influenza infection, optionally selected from Type A, Type B, Type C, and Type D influenza virus infection.

In embodiments, the viral infection is a coronavirus infection. In embodiments, the coronavirus is a betacoronavirus, optionally selected from severe acute respiratory syndrome coronavirus 2 (SARS-CoV-2), SARS-CoV, Middle East respiratory syndrome-corona virus (MERS-CoV), HCoV-HKU1, and HCoV-0C43. In embodiments, the coronavirus is an alphacoronavirus, optionally selected from HCoV-NL63 and HCoV-229E.

The coronavirus is a member of the family Coronaviridae, including betacoronavirus and alphacoronavirus respiratory pathogens that have relatively recently become known to invade humans. The Coronaviridae family includes such betacoronavirus as Severe acute respiratory syndrome coronavirus 2 (SARS-CoV-2), SARS-CoV, Middle East Respiratory Syndrome-Corona Virus (MERS-CoV), HCoV-HKU1, and HCoV-0C43. Alphacoronavirus includes, e.g., HCoV-NL63 and HCoV-229E.

Coronaviruses invade cells through “spike” surface glycoprotein that is responsible for viral recognition of Angiotensin Converting Enzyme 2 (ACE2), a transmembrane receptor on mammalian hosts that facilitate viral entrance into host cells. Zhou et al., A pneumonia outbreak associated with a new coronavirus of probable bat origin. Nature 2020. A new coronavirus infection 2019 (COVID-19), caused by

SARS-CoV-2 is a new virus thought to be originated from the bat. COVID-19 causes severe respiratory distress and this RNA virus strain has been the cause of the recent outbreak that has been declared a major threat to public health and worldwide emergency. Phylogenetic analysis of the complete genome of SARS-CoV-2 revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020

The SARS-CoV-2 is an enveloped, single stranded, RNA virus that encodes a “spike” protein, also known as the S protein, which is a surface glycoprotein that mediates binding to a cell surface receptor; an integral membrane protein; an envelope protein, and a nucleocapsid protein. The S protein, comprising S1 subunit and S2 subunit, is a trimeric class I fusion protein that exists in a prefusion conformation that undergoes a structural rearrangement to fuse the viral membrane with the host-cell membrane. See, e.g., Li, F. Structure, Function, and Evolution of Coronavirus Spike Proteins. Annu. Rev. Virol. 3: 237-261(2016), which is incorporated herein by reference in its entirety. The structure of the SARS-CoV-2 spike protein in the prefusion conformation has been discovered. See Daniel et al., Cryo-EM structure of the SARS-CoV-2 spike in the prefusion conformation. Science, 19 Feb. 2020, which is incorporated herein by reference in its entirety.

Phylogenetic analysis of the complete genome of SARS-CoV-2 (GenBank Accession No.: MN908947) revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020, which is incorporated herein by reference in its entirety.

The SARS-CoV-2 has a spike surface glycoprotein, membrane glycoprotein M, envelope protein E, and nucleocapsid phosphoprotein N. The complete genome of the SARS-CoV-2 coronavirus (29903 nucleotides, single-stranded RNA) is described in the NCBI database as GenBank Reference Sequence: MN908947. The coronavirus protein can be selected from the group consisting of: coronavirus spike protein (GenBank Reference Sequence: QHD43416), coronavirus membrane glycoprotein M (GenBank Reference Sequence: QHD43419), coronavirus envelope protein E (GenBank Reference Sequence: QHD43418), and coronavirus nucleocapsid phosphoprotein E (GenBank Reference Sequence: QHD43423).

In embodiments, the method prevents or mitigates development of acute respiratory distress syndrome (ARDS) in the patient.

In embodiments, the coronavirus is SARS-CoV-2. In embodiments, the patient is afflicted with COVID-19. In embodiments, the patient is afflicted with one or more of fever, cough, shortness of breath, diarrhea, upper respiratory symptoms, lower respiratory symptoms, pneumonia, and acute respiratory syndrome.

In embodiments, the patient is hypoxic. In embodiments, the patient is afflicted with respiratory distress. In embodiments, the method improves oxygenation in the patient. In embodiments, the method prevents or mitigates a transition from respiratory distress to cytokine imbalance in the patient. In embodiments, the method reverses or prevents a cytokine storm. In embodiments, the method reverses or prevents a cytokine storm in the lungs or systemically. In embodiments, the cytokine storm is selected from one or more of systemic inflammatory response syndrome, cytokine release syndrome, macrophage activation syndrome, and hemophagocytic lymphohistiocytosis. In embodiments, the method reverses or prevents excessive production of one or more inflammatory cytokines. In embodiments, the inflammatory cytokine is one or more of IL-6, IL-1, IL-1 receptor antagonist (IL-1ra), IL-2ra, IL-10, IL-18, TNFα, interferon-γ, CXCL10, and CCL7.

In embodiments, the method causes a decrease in viral load in the patient relative to before treatment.

In an aspect, there is provided a method for treating or preventing a viral infection in a subject in need thereof, comprising providing plasma from a donor subject who has recovered from the viral infection, the plasma comprising IgG, IgM and/or IgA antibodies directed against the virus causing the infection and the donor subject having been treated with the recombinant human GM-CSF protein described herein to stimulate production of the antibodies; and administering the plasma to the subject in need thereof. In an aspect, there is provided a method for treating or preventing a viral infection in a subject in need thereof, comprising: administering the recombinant human GM-CSF protein described herein to a donor subject who has recovered from the viral infection; isolating plasma from the donor subject, the plasma comprising IgG, IgM and/or IgA antibodies directed against the virus causing the infection; and administering the plasma to the subject in need thereof.

In embodiments, such methods provide passive immunization against the virus to the subject in need thereof.

In embodiments, the IgG, IgM and/or IgA antibodies specifically bind to a viral antigen. In embodiments, the IgG, IgM and/or IgA antibodies neutralize the virus. In embodiments, the IgG, IgM and/or IgA antibodies prevent or diminish infection of a cell by the virus.

In embodiments, the viral infection is selected from a betacoronavirus infection, optionally selected from severe acute respiratory syndrome coronavirus 2 (SARS-CoV-2), severe acute respiratory syndrome coronavirus (SARS-CoV-1), Middle East Respiratory Syndrome-Corona Virus (MERS-CoV), HCoV-HKU1, and HCoV-0C43 infection. In embodiments, the viral infection is selected from an alphacoronavirus infection, optionally selected from HCoV-NL63 and HCoV-229E infection.

In embodiments, the betacoronavirus infection is severe acute respiratory syndrome (SARS).

In embodiments, the betacoronavirus infection is, or is associated with, coronavirus disease 2019 (COVID-19).

In embodiments, the viral infection is an influenza infection, optionally selected from Type A, Type B, Type C, and Type D influenza virus infection. In embodiments, the influenza infection is pandemic 2009 influenza A (H1N1) or avian influenza A (H5N1).

In embodiments, donor subject has tested positive for the viral infection prior to recovery. In embodiments, the donor subject has resolution of viral infection symptoms prior to donation. In embodiments, the donor subject has tested positive for antibodies directed against the virus using a serological test. In embodiments, the donor subject demonstrates measurable neutralizing antibody titers. In embodiments, the neutralizing antibody titers are at least about 1:160. In embodiments, the plasma is isolated from a blood sample from the donor subject. In embodiments, the plasma is isolated via plasmapheresis. In embodiments, the plasma comprises a therapeutically effective amount of the IgG, IgM and/or IgA antibodies directed against the virus causing the infection.

### Combination Therapy and Additional Therapeutic Agents

In various embodiments, the pharmaceutical composition of the present invention is co-administered in conjunction with additional agent(s). Co-administration can be simultaneous or sequential.

In one embodiment, the additional therapeutic agent and the GM-CSF of the present invention are administered to a subject simultaneously. The term “simultaneously” as used herein, means that the additional therapeutic agent and the GM-CSF are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the GM-CSF can be by simultaneous administration of a single formulation (e.g., a formulation comprising the additional therapeutic agent and the GM-CSF composition) or of separate formulations (e.g., a first formulation including the additional therapeutic agent and a second formulation including the GM-CSF composition).

Co-administration does not require the therapeutic agents to be administered simultaneously, if the timing of their administration is such that the pharmacological activities of the additional therapeutic agent and the GM-CSF overlap in time, thereby exerting a combined therapeutic effect. For example, the additional therapeutic agent and the targeting moiety, the GM-CSF composition can be administered sequentially. The term “sequentially” as used herein means that the additional therapeutic agent and the GM-CSF are administered with a time separation of more than about 60 minutes. For example, the time between the sequential administration of the additional therapeutic agent and the GM-CSF can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the GM-CSF being administered. Either the additional therapeutic agent or the GM-CSF composition may be administered first.

Co-administration also does not require the therapeutic agents to be administered to the subject by the same route of administration. Rather, each therapeutic agent can be administered by any appropriate route, for example, parenterally or non-parenterally.

In some embodiments, the GM-CSF described herein acts synergistically when co-administered with another therapeutic agent. In such embodiments, the targeting moiety, the GM-CSF composition and the additional therapeutic agent may be administered at doses that are lower than the doses employed when the agents are used in the context of monotherapy.

In some embodiments, the additional therapeutic agent is an anti-viral drug.

In some embodiments, the additional therapeutic agent is selected from drugs including antivirals such as remdesivir, favipiravir, oseltamivir, baloxavir, galidesivir, amprenavir, tipranavir, saquinavir, nelfinavir, indinavir, darunavir, atazanavir, emetine, lopinavir and/or ritonavir, arbidol and lopinavir/ritonavir, and/or ribavirin, darunavir and cobicistat, and/or IFN-beta-1 b, B-D-N4-hydroxycytidine (NHC) such as EIDD-1931 or EIDD-2801 or EIDD-2801; immunomodulators such as glucocorticoids, IFN-α 2a, IFN-α 2b, IFN-b, pegylated IFN-g, baricitinib, sirolimus, clazakizumab, canakinumab, XPro1595, tocilizumab, sarilumab, siltuximab, adalimumab, eculizumab, ivermectin, anakinra, prezcobix, xiyanping, fingolimod, methylprednisolone, leronlimab, thalidomide, MK-2206, nicolasamide, nitazoxamide, chloroquine or hydroxychloroquine; antibiotics such as carrimycin, brilacidin, azithromycin, valinomycin, angiotension inhibitors/antagonists like rhACE2/GSK2586881/APN01, losartan, eprosartan, telmisartan, valsartan; serine protease inhibitor including camostat mesylate, nafamostat other drugs such as bromhexine, aprotinin, chlorpromazine, zotatifin, methotrexate, lenalidomide, anti-VEGF-A and Intravenous Immunoglobulin (IVIG). For instance, in embodiments, any of these additional therapeutic agents find use in the context of a SARS-CoV-2 infection.

In some embodiments, the additional therapeutic agent is selected from favipiravir, laninamivir octanoate, peramivir, zanamivir, oseltamivir phosphate, baloxavir marboxil, umifenovir, urum in amantadine hydrochloride, rimantadine hydrochloride, adapromine, LASAG/BAY81-87981, celecoxib, etanercept, metform in, gemcitabine, dapivirine, trametinib, lisinopril, naproxen, nalidixic acid, dorzolamide, ruxolitinib, midodrine, diltiazem; statins including atorvastatin, nitazoxanide; PPAR antagonists including gemfibrozil. For instance, in embodiments, any of these additional therapeutic agents find use in the context of a influenza infection.

### Sequences

SEQ ID NO: 1 is wild type GM-CSF.
APARSPSPSTQPWEHVNAIQEARRLLNLSRDTAAEMNETVEVIS
EMFDLQEPTCLQTRLELYKQGLRGSLTKLKGPLTMMASHYKQHC
PPTPETSCATQIITFESFKENLKDFLLVIPFDCWEPVQE.
SEQ ID NO: 2 is sargramostim.
APARSPSPSTQPWEHVNAIQEALRLLNLSRDTAAEMNETVEVIS
EMFDLQEPTCLQTRLELYKQGLRGSLTKLKGPLTMMASHYKQHC
PPTPETSCATQIITFESFKENLKDFLLVIPFDCWEPVQE.

### Definitions

The following definitions are used in connection with the invention disclosed herein. Unless defined otherwise, all technical and scientific terms used herein have the same meaning as commonly understood to one of skill in the art to which this invention belongs.

An “effective amount,” when used in connection with an agent effective for the treatment of a coronavirus infection is an amount that is effective for treating or mitigating a coronavirus infection.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

This invention is further illustrated by the following non-limiting examples.

### EXAMPLES

### Example 1: List of Fermentation Supplements

Production fermentation was executed incorporating supplementation with key components found in the complex materials, Bacto-Peptone and Yeast Extract. The list of fermentation supplements included MgSO₄, KH₂PO₄, CaCl₂), adenine, MEM Vitamin Solution and YNB Trace Elements solution. There was a notable increase in biomass and productivity in fermentations carried out in the presence of all supplements and only the Trace Elements Solution, indicating that the Trace Elements Solution contains the key component for increasing recombinant human (rhu) GM-CSF productivity and culture biomass. There are six elements in the trace elements solution: copper, molybdate, zinc, iron, boric acid and manganese. To identify which of the elements were responsible for increased productivity and biomass, the six trace elements screened individually in production fermentation in concentrations consistent with YNB Trace Elements Solution, as indicated in Table 1 (final concentration in the fermenter for each element screened).

Table 1 lists the various trace elements and their concentrations tested in the fermenter during the manufacturing process:

| | Concentration | Concentration |
|---|---|---|
| | in Fermenter | in Fermenter |
| Material | (g/L) | (μM) |
| Cupric Sulfate, | 0.0004 | 1.6 |
| (5) H₂O | | |
| Sodium | 0.0020 | 8.3 |
| Molybdate, (2) | | |
| H₂O | | |
| Zinc Sulfate, | 0.0040 | 13.9 |
| (7) H₂O | | |
| Ferric | 0.0020 | 7.4 |
| Chloride, (6) | | |
| H₂O | | |
| Boric Acid | 0.0050 | 80.9 |
| Manganese | 0.0040 | 23.7 |
| Sulfate, | | |
| (1)H₂O | | |

### Example 2: Biochemical Assays of Trace Elements Supplementation

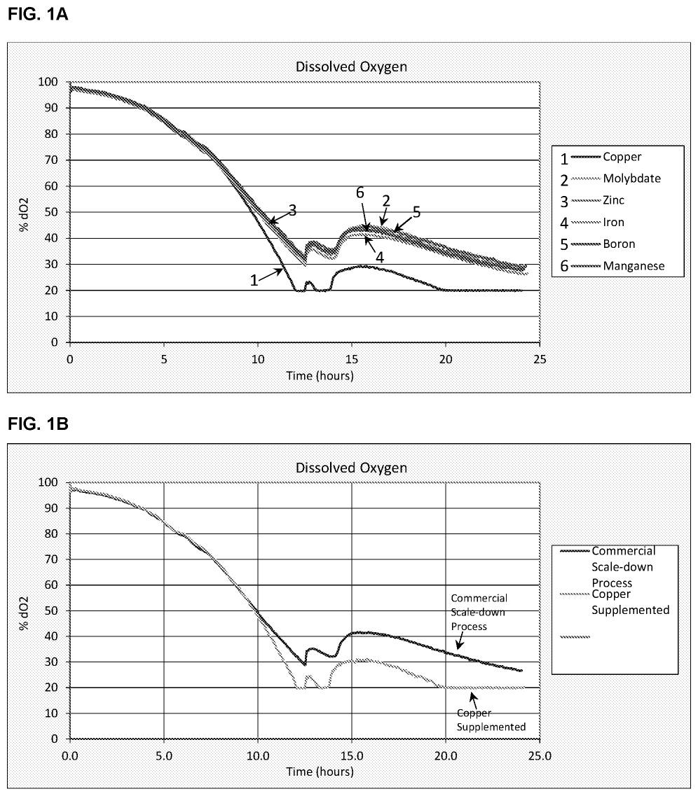

Dissolved Oxygen Profile: The dissolved oxygen level is routinely monitored as a process parameter during production fermentation and serves as a surrogate for yeast culture oxygen uptake, indicating yeast culture growth. Dissolved oxygen profiles are shown for production fermentations carried out in the presence of each individual trace element (FIG. 1A). The yeast culture oxygen uptake was significantly greater in the copper (copper sulfate/CuSO4) supplemented batches resulting in a decrease of the dissolved oxygen levels. A dissolved oxygen cascade control strategy was used to prevent the dissolved oxygen falling below inhibitory levels.

In FIG. 1B, the dissolved oxygen profile for production fermentations carried out in the presence of copper supplementation was compared to the profile of the commercial scale-down process (no supplementation). The results demonstrate a significant difference in oxygen demand in yeast cultures in the presence of copper supplementation.

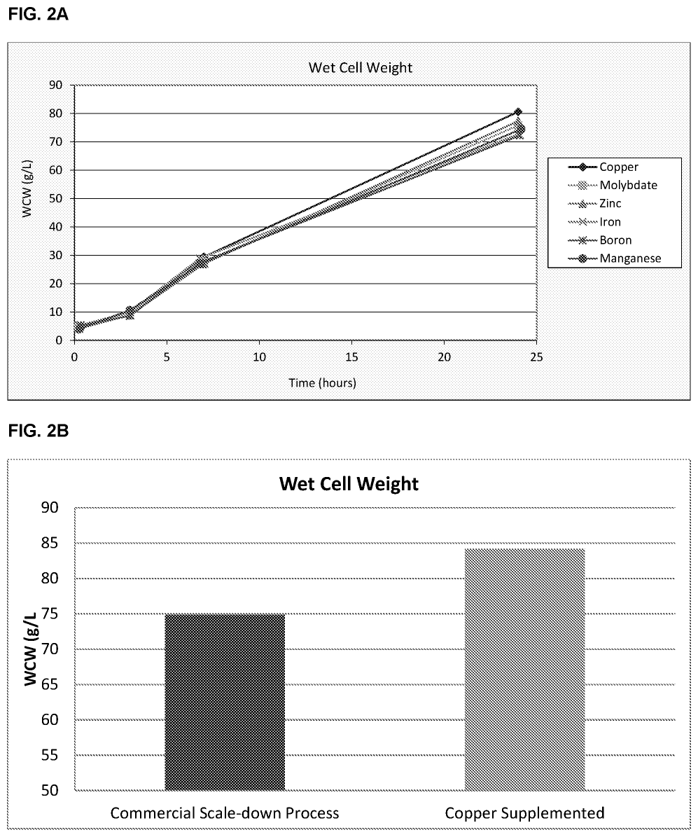

Wet Cell Weight Profile: Yeast culture biomass was assessed as culture wet cell weight (WCW). WCW was determined by centrifugation of 20 mL of cell broth in a pre-weighed 50 mL centrifuge tube. Supernatant was aspirated off, and the tube was weighed again to calculate the WCW for each production fermentation batch. WCW is shown for production fermentations carried out in the presence of each individual trace element (FIG. 2A), with the highest biomass resulting in the presence of copper supplementation. When copper supplementation was compared to the commercial scale-down process (no supplementation), biomass was notably higher in the copper supplemented fermentation than the commercial scale-down fermentation (FIG. 2B).

### Example 3: Comparison of Recombinant Human GM-CSF Titers and Glycoforms

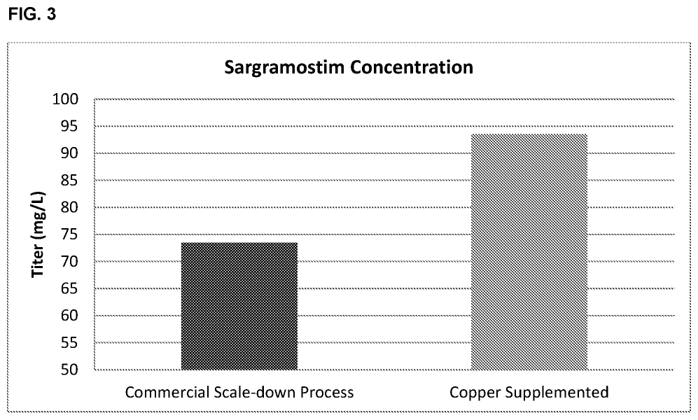

Reverse-phase HPLC was used for determination of recombinant human (rhu) GM-CSF concentrations in test samples using a C18 column in an acetonitrile gradient with constant composition of 0.2M sodium chloride maintained throughout the gradient program. Trifluoroacetic acid (TFA) was used as an ion pairing reagent (0.1% by volume in each mobile phase solvent). Test sample rhu GM-CSF concentration results were interpolated from a six-level external standard calibration curve prepared from a GM-CSF reference standard. FIG. 3 illustrates a notable increase in rhu GM-CSF concentration compared to the commercial scale-down process (with no supplementation).

The reverse phase HPLC procedure used to determine rhu GM-CSF concentration resolves rhu GM-CSF glycosylated variants into three main glycoform groups across the C18 column. Four peaks of interest were integrated and quantitated; the composition of each is described below:
Peak 1=GM-CSF related impurity (oxidation).
Note: in samples prior to C4 Purification, a hyperglycosylated peak is present that masks the true peak 1.
Peak 2=N- and (N+O) linked glycoforms
Peak 3=O-linked glycoforms
Peak 4=Non-glycosylated GM-CSF
   Table 2 shows that the glycosylation variants (percentage peaks 2-4), indicative of product quality, from fermentations carried out in the presence copper supplementation are comparable to the historical means of the commercial process. Percentage of glycosylation variants obtained in the presence of copper supplementation are within a 95% tolerance interval that covers 99.73% of the full production history of commercial rhu GM-CSF, indicating no impact of copper supplementation on GM-CSF glycoforms or product quality attributes.

Table 2 illustrates the glycoform profiles (shown as percent peaks) of the recombinant human GM-CSF obtained by the exogenous copper-supplemented fermentation process as compared to the historical commercial scale-down process. This table compares the percent peaks of the copper supplemented fermenter to the historical commercial mean and the commercial acceptance criteria.

TABLE 2
Sargramostim Glycoform Comparability

| | Peak 2% | Peak 3% | Peak 4% |
|---|---|---|---|
| Copper Supplementation | 26.0 | 21.0 | 52.9 |
| Historical Commercial | 27.8 | 21.3 | 51.0 |
| Mean | | | |
| Lower Limit of 95/99.7% | 23.9 | 18.5 | 47.7 |
| Tolerance Interval | | | |
| Upper Limit of 95/99.7% | 31.7 | 24.1 | 54.3 |
| Tolerance Interval | | | |

### Example 4: Comparison of In-Process and Routine Release Testing Results of Trace Elements Supplementation

Analysis of the data was performed on the C4 Purification (Table 3), C18 (Table 4), and Bulk Drug Substance (BDS). The data demonstrated that the production fermentation supplemented with copper produced material that is comparable to material produced by the current commercial manufacturing process.

The key indicator for product quality of the protein through downstream operations is glycoform ratio as determined by the T-0075 assay. Peaks 2, 3, and 4 represent the glycosylated variants of sargramostim, while peak 1 is hyperglycosylated impurity. Peak 1 is removed in the C4 Purification unit operation. In-Process and BDS glycoform results for the CuSO4 supplemented BDS process validation (CuSO4 PV) are comparable to commercial in-process and BDS lots (BDS 6-8).

Table 3 illustrates Glycoform Ratio Comparability Summary. Table 3 illustrates the C4 Purification glycoform ratio comparability summary for copper-supplemented fermentation process as compared to the historical commercial process. The batch numbers listed in Table 3 and Table 4 are associated with the C4 purification PV runs.

TABLE 3
C4 Purification Glycoform Ratio Comparability Summary

| | | | | | C4 | | |
|---|---|---|---|---|---|---|---|
| | | | | Comparability | Purification | | |
| Process | Process | Mean | Mean | Acceptance | Batch | | |
| Step | Parameter | (Historical) | (PV) | Criteria | Number | Result | Comparable |
| C4 | Peak 1 | 2.4% | 2.4% | 1.0-3.8% | B26131 | 2.5% | Yes |
| Purification | | | | | B26132 | 2.4% | Yes |
| (M/N | | | | | B26133 | 2.4% | Yes |
| 12834) | | | | | B26134 | 2.4% | Yes |
| | Peak 2 | 28% | 29% | 25-31% | B26131 | 29% | Yes |
| | | | | | B26132 | 29% | Yes |
| | | | | | B26133 | 28% | Yes |
| | | | | | B26134 | 28% | Yes |
| | Peak 3 | 22% | 22% | 18-25% | B26131 | 23% | Yes |
| | | | | | B26132 | 21% | Yes |
| | | | | | B26133 | 22% | Yes |
| | | | | | B26134 | 22% | Yes |
| | Peak 4 | 48% | 47% | 45-51% | B26131 | 46% | Yes |
| | | | | | B26132 | 47% | Yes |
| | | | | | B26133 | 48% | Yes |
| | | | | | B26134 | 48% | Yes |

Table 4 illustrates the C18 Purification glycoform ratio comparability summary for copper-supplemented fermentation process as compared to the historical commercial process.

TABLE 4
C18 Purification Glycoform Ratio Comparability Summary

| | | | | Comparability | C18 | | |
|---|---|---|---|---|---|---|---|
| Process | Process | Mean | Mean | Acceptance | Purification | | |
| Step | Parameter | (Historical) | (PV) | Criteria | Batch Number | Result | Comparable |
| C18 | Peak 1 | 2.4% | 2.7% | 1.0-3.9% | B26135 | 2.7% | Yes |
| Purification | | | | | B26136 | 2.6% | Yes |
| (M/N | Peak 2 | 28% | 30% | 25-31% | B26135 | 29% | Yes |
| 12836) | | | | | B26136 | 30% | Yes |
| | Peak 3 | 22% | 22% | 18-25% | B26135 | 22% | Yes |
| | | | | | B26136 | 22% | Yes |
| | Peak 4 | 48% | 46% | 45-51% | B26135 | 46% | Yes |
| | | | | | B26136 | 46% | Yes |

Table 5: illustrates the BDS glycoform ratio comparability summary for copper-supplemented fermentation process as compared to the historical commercial scale-down process.

TABLE 5
BDS Glycoform Ratio Comparability Summary

| | | | | Compa- | | |
|---|---|---|---|---|---|---|
| | BDS | | | rability | | |
| Process | Batch | Process | Mean | Acceptance | | Com- |
| Step | Number | Parameter | (Historical) | Criteria | Result | parable |
| BDS | CuSO4 | Peak 1 | 2.2% | 1.0-3.5% | 2.5% | Yes |
| (M/N | PV | Peak 2 | 28% | 26-31% | 29% | Yes |
| 12840) | | Peak 3 | 22% | 19-25% | 22% | Yes |
| | | Peak 4 | 48% | 45-50% | 46% | Yes |

### Example 5: Comparison of BDS Release Testing of Trace Elements Supplementation

The results for the BDS release testing on the 3 commercial BDS and the 1 process validation BDS all passed current specification criteria for BDS release. All results support the comparability of the sargramostim protein produced during the copper-supplemented process validation runs (CuSO4 PV) with results from the historical commercial runs (BDS 6-8).

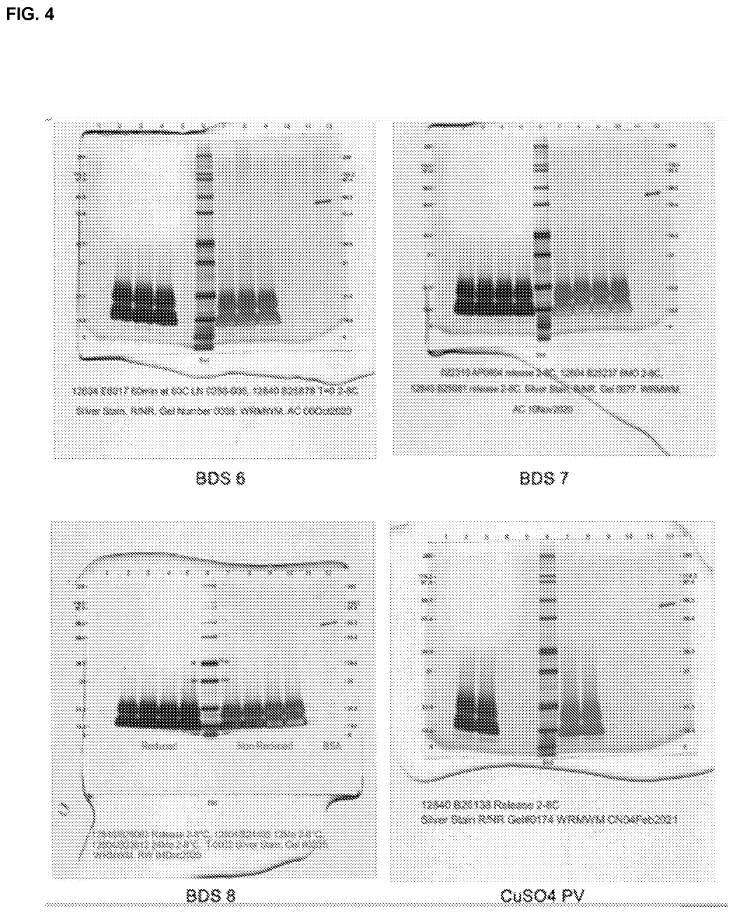

FIG. 4 illustrates the results from SDS-PAGE-Silver Stain (T-0002) assay that was used to evaluate impurities in sargramostim BDS due to protein degradation or non-product contamination. Test results for impurities for the CuSO4 batch at BDS (CuSO4 PV) are comparable to levels in commercial BDS batches 6-8.

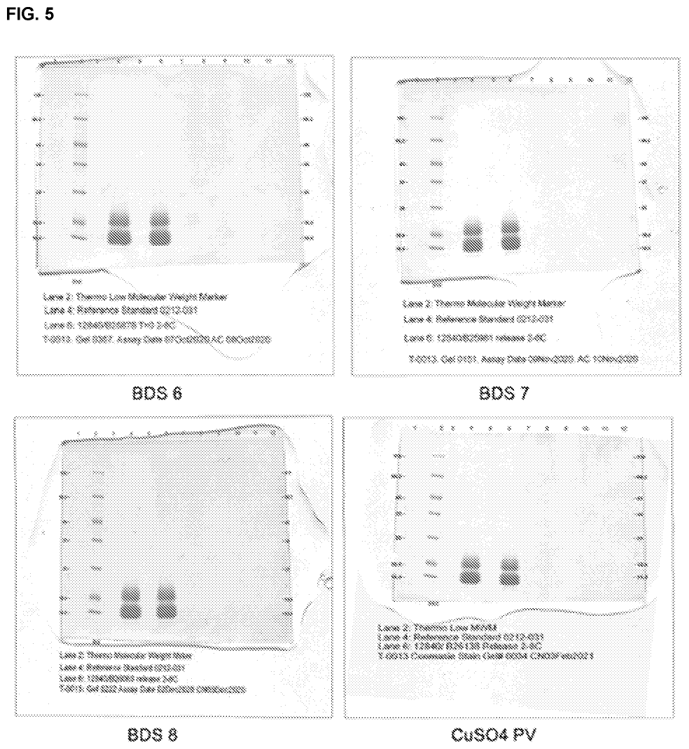

FIG. 5 illustrates the results from densitometry testing (T-0013) that was performed to evaluate the level of protein purity of the sargramostim BDS. Test results for protein purity of the CuSO4 batch at BDS (CuSO4 PV) are comparable to levels in commercial BDS batches 6-8.

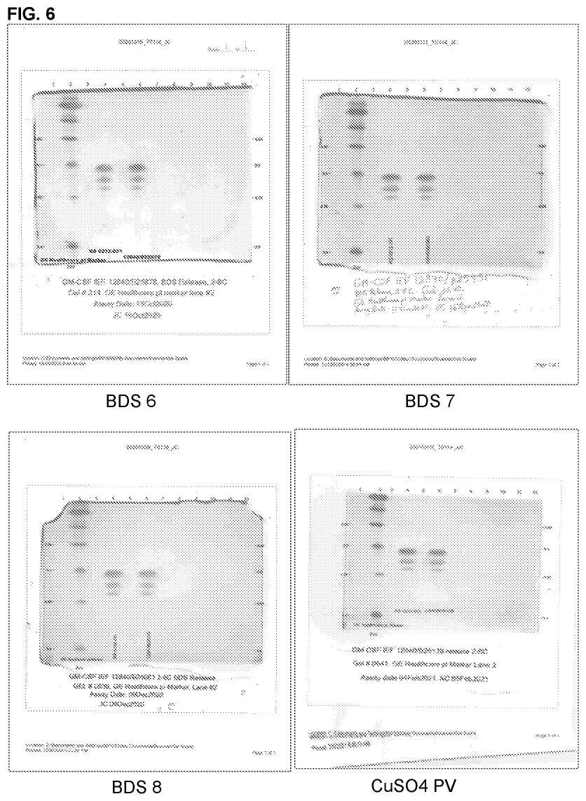

FIG. 6 illustrates the results from isoelectric focusing (T-0114) which was used to determine the identity of the sargramostim BDS. Isoelectric Focusing test results for the CuSO4 batch at BDS (CuSO4 PV) are comparable to results in commercial BDS batches 6-8.

Table 6 and Table 7 further provides a summary of the BDS release testing results.

TABLE 6
BDS Release Test Results

| | Test | |
|---|---|---|
| Test | Description | Acceptance Criteria |
| T-0002 | SDS-PAGE | The mobility of the 3 bands of the test sample must correspond to the |
| | | molecular weights based on comparison to MW markers and a rhu GM- |
| | | CSF Ref. Std. run on the same gel. |
| | | Test Sample displays no extra bands that are present in Ref. Std. |
| T-0013 | Densitometry | Protein purity is ≥99% by area |
| T-0019 | pH | 7.2-7.6 |
| T-0023 | ACC | Clear, colorless to pale straw liquid |
| T-0091 | Bioassay | 4.0-6.9 × 10⁶ IU/mg |
| T-0108 | Monosaccharide | 3.63-5.22 moles of mannose/mole of sargramostim |
| | | 0.326-0.433 moles of N-acetylglucosamine/mole of sargramostim |
| T-0114 | Isoelectric | Major species migrates at pl 5.2 +/− 0.2 with no more than 3 minor species |
| | Focusing | evident in the pl range 4.5 to 5.2 |
| T-0154 | SE-HPLC | ≤1.0% for higher molecular weight component. |
| T-0315 | UV Spec | 5.0-8.3 mg/mL |
| | Scan | |
| T-0323 | Peptide | Ala1: 60-85% |
| | Mapping | Ala3: 15-40% |
| | | Arg4 ≤2% |
| | | Ser5: ≤5% |
| T-3007 | Endotoxin | ≤1.25 EU/mg |
| T-3011 | Micro Content | <1 CFU/ml |

TABLE 7
BDS Release Test Results

| | BDS TEST RESULTS | | | | |
|---|---|---|---|---|---|
| | | | | CuSO4 PV | |
| Test | 6 (B25878) | 7 (B25981) | 8 (B26063) | (B26138) | Comparable |
| T-0002 | Pass | Pass | Pass | Pass | Yes |
| T-0013 | 100.00 | 99.60 | 99.47 | 99.46 | Yes |
| T-0019 | 7.42 | 7.44 | 7.43 | 7.47 | Yes |
| T-0023 | Pass | Pass | Pass | Pass | Yes |
| T-0091 | 6.0*10⁶ IU/mg | 6.0*10⁶ IU/mg | 5.8*10⁶ IU/mg | 6.2*10⁶ IU/mg | Yes |
| T-0108 | 4.47 | 4.65 | 4.92 | 4.58 | Yes |
| | 0.352 | 0.386 | 0.404 | 0.393 | Yes |
| T-0114 | Pass | Pass | Pass | Pass | Yes |
| T-0154 | <0.1 | <0.1 | <0.1 | <0.1 | Yes |
| T-0315 | 6.37 | 6.40 | 6.63 | 6.68 | Yes |
| T-0323 | Ala1: 71.2 | Ala1: 71.0 | Ala1: 71.4 | Ala1: 69.8 | Yes |
| | Ala3: 28.8 | Ala3: 29.0 | Ala3: 28.6 | Ala3: 30.2 | |
| | Arg4: <0.57 | Arg4: <0.57 | Arg4: <0.57 | Arg4: <0.57 | |
| | Ser5: <1.43 | Ser5: <2.29 | Ser5: <1.43 | Ser5: <2.29 | |
| T-3007 | <0.05 EU/mg | <0.05 EU/mg | <0.05 EU/mg | <0.05 EU/mg | Yes |
| T-3011 | 0 CFU/mL | 0 CFU/mL | 0 CFU/mL | 0 CFU/mL | Yes |

### Example 7: Product Protein Characterization with the CuSO4 Supplemented Manufacturing Process as Compared to the Approved Commercial Process

To ensure the product protein produced with the CuSO4 manufacturing process is comparable to the approved commercial process, additional characterization of the product and process were performed. For the product characterization, assays were performed that provide detailed evaluation of the protein composition and structure. Results support the comparability of the sargramostim protein produced during the process validation runs (CuSO4 PV batch, CuSO4 PV) with results from commercial BDS runs BDS 6-8. Table 8 provides a summary of the product characterization results.

TABLE 8
Product Characterization Results

| Test Method | Purpose | Observation | Result |
|---|---|---|---|
| Elisa | Evaluate the removal of | Residual process components removal | PV batch |
| | Residual Process | at BDS were comparable across the 4 | comparable |
| | Components (RPC) | batches and full historical data set. | to 3 |
| | | | commercial |
| | | | batches |
| Tryptic peptide | Evaluate C-terminal | The 3 major C-terminal peptides were | PV batch |
| map | proteolysis | comparable by retention time and | comparable |
| | | normalized percent area across the 4 | to 3 |
| | | batches evaluated confirming an intact | commercial |
| | | C-terminus. | batches |
| Low pH Glu C | Determines correct pairing | Expected masses of disulfide bridged | PV batch |
| peptide map with | of di-sulfides | peptides were confirmed and | comparable |
| mass spec | | comparable across the 4 batches | to 3 |
| analysis | | confirming correct disulfide pairing. | commercial |
| | | | batches |
| Low pH Glu C | Determines size of N-linked | Percent N-linked glycosylation at | PV batch |
| peptide map | chain at position N27 (site | position N27 was comparable across | comparable |
| (+/−PNGase) | occupancy) | the 4 batches. | to 3 |
| | | | commercial |
| | | | batches |
| Neutral pH Glu C | Determination of size of O- | Percent O-linked glycosylation was | PV batch |
| peptide map | linked sugar chain (site | comparable across the 4 batches. | comparable |
| without alpha- | occupancy) | | to 3 |
| mannosidase | | | commercial |
| | | | batches |
| Neutral pH Glu C | Methionine Oxidation at | Percent oxidation at Methionine 79 was | PV batch |
| peptide map | position 79 | comparable across the 4 batches. | comparable |
| | | | to 3 |
| | | | commercial |
| | | | batches |
| Intrinsic | Determination of tertiary | Tertiary structure and thermodynamic | PV batch |
| Fluorescence | structure and melting | stability (thermal unfolding) were | comparable |
| | transition | comparable by spectra and melting | to 3 |
| | | temperatures (Tₘ) across the 4 batches | commercial |
| | | evaluated. | batches |
| Circular | Determination of secondary | The 4 batches had comparable CD | PV batch |
| Dichroism | structure, melting temp., and | scans, melting temperatures (Tₘ) and | comparable |
| | onset of protein unfolding. | onset of protein unfolding (Tonset) | to 3 |
| | | | commercial |
| | | | batches |
| MALDI-TOF | Identification of Intact protein | MALDI-TOF profiles and N-&-O-linked | PV batch |
| | and N-&O- linked glycan | glycan structures were comparable | comparable |
| | structure | across the 4 batches. | to 3 |
| | | | commercial |
| | | | batches |
| Proteomic LC- | Globally identify and | All batches contain >99% sargramostim | PV batch |
| MS/MS HCP | quantitate low abundance | and the low HCP identities and | comparable |
| analysis | host cell proteins | quantities are comparable across the 4 | to 3 |
| | | batches | commercial |
| | | | batches |
| ICP-MS | Determination of elemental | All results correspond to less than the | PV batch |
| Quantitative | impurity levels as defined by | Permitted Daily Exposure for a | comparable |
| Screen Test | ICH Q3D for parenteral | parenteral drug product as described in | to 3 |
| | products. | ICH Q3D. | commercial |

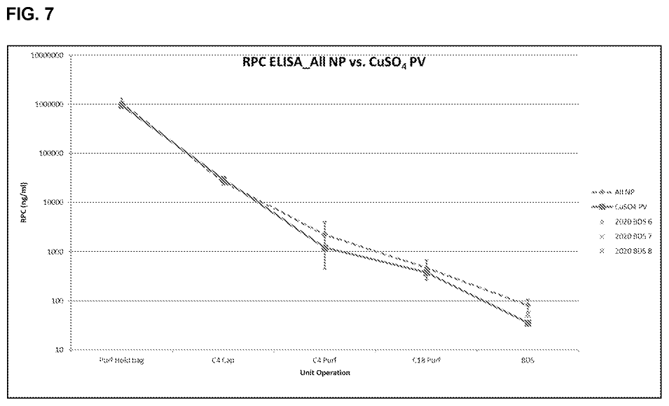

Process characterization consisted of evaluating the removal of residual process components (RPC) throughout downstream operations. Sample analysis of the CuSO4 PV batch (CuSO4 PV) show RPC removal throughout the purification process. Levels of RPC for the CuSO4 batch at BDS were comparable to levels of both recent and all historical batches. Results support that the level of RPC removal for the CuSO4 supplemented process was comparable to the current manufacturing process. Results are shown in FIG. 7 and Table 9.

TABLE 9
Residual Process Components (RPC) Summary

| | Unit Operation | | | | |
|---|---|---|---|---|---|
| | Purf Hold | | | | |
| | bag | C4 Cap | C4 Purf | C18 Purf | BDS |
| Data Set | (ng/mL) | (ng/mL) | (ng/mL) | (ng/mL) | (ng/mL) |
| Full | 1086023 | 26359 | 2232 | 472 | 81 |
| Historical | | | | | |
| (average) | | | | | |
| CuSO4 PV | 944090 | 29743 | 1215.4 | 376.44 | 35.125 |
| (B26138) | | | | | |
| BDS 6 | | | | | 51.805 |
| (B25878) | | | | | |
| BDS 7 | | | | | 36.17 |
| (B25981) | | | | | |

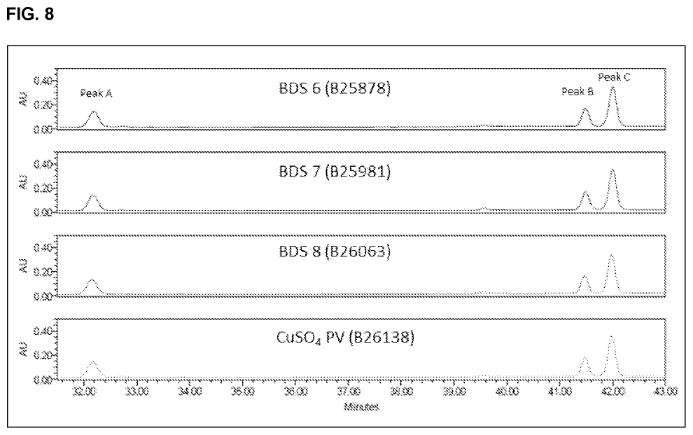

C-term inal analysis was performed utilizing a tryptic peptide map (TCPK-Trypsin) 1. rhuGM-CSF is enzymatically digested with trypsin and reduced. The generated peptides are separated by RP-HPLC. The three major C-terminal peptides are analyzed by retention time and quantitated by normalized % area. Results for C-terminal analysis show comparability between the CuSO4 batch at BDS (CuSO4 PV) and the commercial BDS batches (BDS 6-8). Results are shown in FIG. 8 and Table 10.

TABLE 10
Reduced Tryptic map (A220) Summary

| | Normalized % Area | | | Retention Times (min) | | |
|---|---|---|---|---|---|---|
| Sample: | Peak A % | Peak B % | Peak C % | Peak A | Peak B | Peak C |
| BDS 6 | 27.4 | 22.2 | 50.4 | 32.2 | 41.5 | 42.0 |
| (B25878) | | | | | | |
| BDS 7 | 27.0 | 22.0 | 50.9 | 32.2 | 41.5 | 42.0 |
| (B25981) | | | | | | |
| BDS 8 | 26.8 | 22.1 | 51.1 | 32.2 | 41.5 | 42.0 |
| (B26063) | | | | | | |
| CuSO4 | 27.0 | 22.2 | 50.8 | 32.2 | 41.5 | 42.0 |
| PV | | | | | | |
| (B26138) | | | | | | |

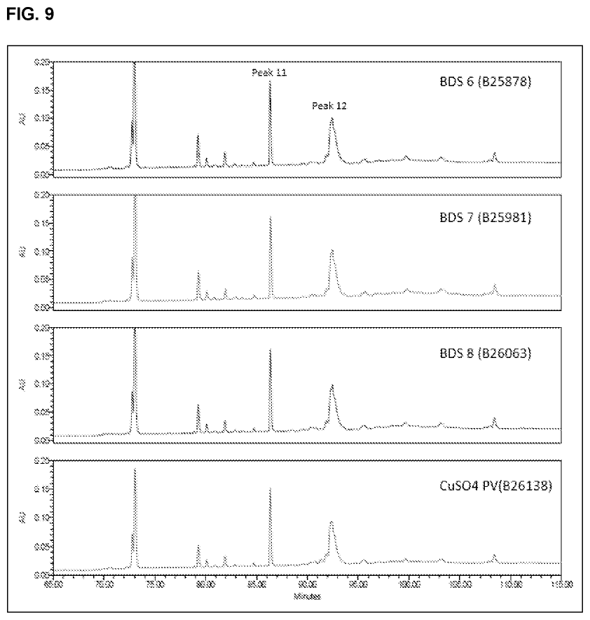

The disulfide bridge pairing is determined by the low pH Glu-C peptide map. The low pH is necessary to prevent disulfide rearrangement. The two major peaks 11 and 12 contain the expected disulfide bridged peptides (G7-8=G10 and G9=G11-13/G9=G12-13, respectively). Peptide fragments were confirmed by mass spec analysis. Disulfide pairing results show comparability between the CuSO4 batch at BDS batch (CuSO4 PV) and the commercial BDS batches (BDS 6-8). Results are shown in Table 11 and FIG. 9.

TABLE 11
Theoretical and Experimental results for disulfide peptide fragments

| | | Peptide | Theoretical | Experimental |
|---|---|---|---|---|
| Sample | Peak | Fragment | Mass (Da) | Mass (Da) |
| BDS 6 (B25878) | Peak 11 | G7-8 = G10 | 3037.44 | 3034.7 |
| | Peak 12 | G9 = G11-13 | 6509.59 | 6516.3 |
| | | G9 = G12-13 | 6018.05 | 6015.3 |
| BDS 7 (B25981) | Peak 11 | G7-8 = G10 | 3037.44 | 3035.4 |
| | Peak 12 | G9 = G11-13 | 6509.59 | 6513.9 |
| | | G9 = G12-13 | 6018.05 | 6014.5 |
| BDS 8 (B26063) | Peak 11 | G7-8 = G10 | 3037.44 | 3034.7 |
| | Peak 12 | G9 = G11-13 | 6509.59 | 6514.5 |
| | | G9 = G12-13 | 6018.05 | 6017.4 |
| CuSO4 PV | Peak 11 | G7-8 = G10 | 3037.44 | 3034.8 |
| (B26138) | Peak 12 | G9 = G11-13 | 6509.59 | 6514.7 |
| | | G9 = G12-13 | 6018.05 | 6015.0 |

Size of N-linked chain at site N27 (site occupancy at N27) was determined by the low pH Glu-C peptide map which was performed removing both N- and O-linked oligosaccharides (with PNGase and alpha-mannosidase respectively). In removing the N-linked oligosaccharides the enzyme PNGase converts the asparaginyl N-linked residue into an aspartyl residue, and the resulting deamidated fragments can be quantitated by RP-HPLC. This method was used to determine total % N-linked glycosylation at position 27 using the following formula:

$$\begin{matrix}\% N ‐ linked \\ glycosylation\end{matrix} = \frac{{Area}_{deamidated\_G 3} + {Area}_{deamidated\_G 4}}{{Area}_{G 3} + {Area}_{G 4} + {Area}_{deamidated\_G 3} + {Area}_{deamidated\_G 4}} ⨯ 100$$

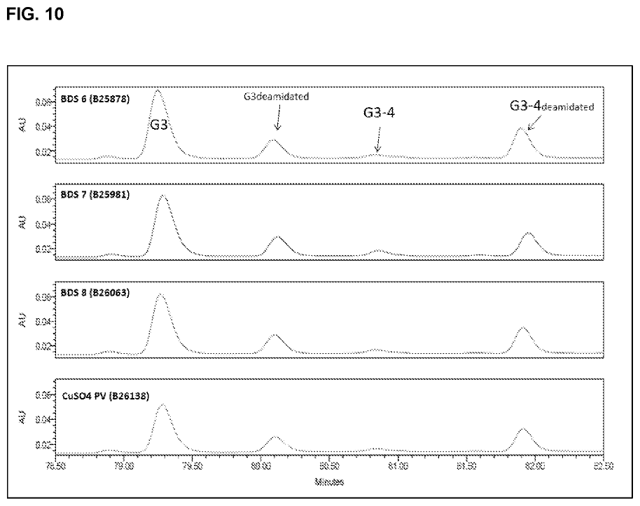

Total % N-linked glycosylation at position 27) show comparability between the CuSO4 batch at BDS (CuSO4 PV) are comparable to results in commercial BDS batches 6-8 and the commercial BDS batches (BDS 6-8). Results are shown in Table 12 and FIG. 10 (Low pH Glu C peptide map chromatogram (78.5-82.5 min) containing the peptides G3-4 and deamidated fragments).

TABLE 12
Percent N-linked glycosylation

| | G3 | | G3_{Deamidated*} | | G3-4 | | (G3-4)_{Deamidated*} | | | | |
|---|---|---|---|---|---|---|---|---|---|---|---|
| | Ret | | Ret | | Ret | | Ret | | % Glycosylated | | |
| LOT | Time | Area | Time | Area | Time | Area | Time | Area | G3 | G3-4 | Total |
| BDS 6 | 79.2 | 568301 | 80.1 | 140705 | 80.8 | 39775 | 81.901 | 215765 | 20% | 84% | 37% |
| (B25878) | | | | | | | | | | | |
| BDS 7 | 79.3 | 507369 | 80.1 | 144471 | 80.9 | 46141 | 81.951 | 165571 | 22% | 78% | 36% |
| (B25981) | | | | | | | | | | | |
| BDS 8 | 79.3 | 497963 | 80.1 | 141445 | 80.8 | 42823 | 81.914 | 183747 | 22% | 81% | 38% |
| (B26063) | | | | | | | | | | | |
| CuSO4 | 79.3 | 385592 | 80.1 | 116632 | 80.8 | 31860 | 81.916 | 164348 | 23% | 84% | 40% |
| PV | | | | | | | | | | | |
| (B26138) | | | | | | | | | | | |

Quantitation of total O-glycosylated glycoforms was evaluated by comparison of the Glu-C peptide map without the use of alpha-mannosidase. The total O-linked glycosylation chain size (site occupancy) was determined by the total area of the O-linked glycoform peaks compared to the unmodified area expressed as a percent using the following formula.

$$\% O ‐ l inked glycosylation = \frac{{Area}_{glycosylated}}{{Area}_{glycosylated} + {Area}_{non ‐ glycosylated}} ⨯ 100$$

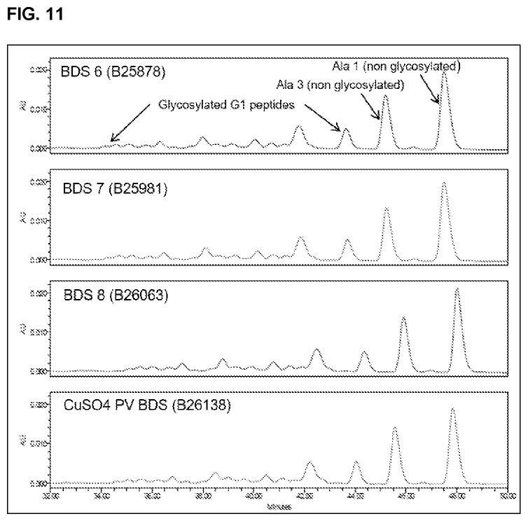

The total O-linked glycosylation chain size (site occupancy) show comparability between the CuSO4 batch at BDS (CuSO4 PV) are comparable to results in commercial BDS batches 6-8 batch (CuSO4 PV) and the commercial BDS batches (BDS 6-8). Results are shown in Table 13 and FIG. 11 (Glu C peptide map without a-mannosidase chromatograms).

TABLE 13
Percent O-linked glycosylation

| | BDS 6 | BDS 7 | BDS 8 | CuSO4 PV |
|---|---|---|---|---|
| | (B25878) | (B25981) | (B26063) | (B26138) |
| Total Area | 667961 | 660441 | 652372 | 635161 |
| _{Glycosylated} | | | | |
| Total Area | 832068 | 809972 | 811200 | 796943 |
| _{Unmodified} | | | | |
| % Glycosylation | 44.5 | 44.9 | 44.6 | 44.4 |

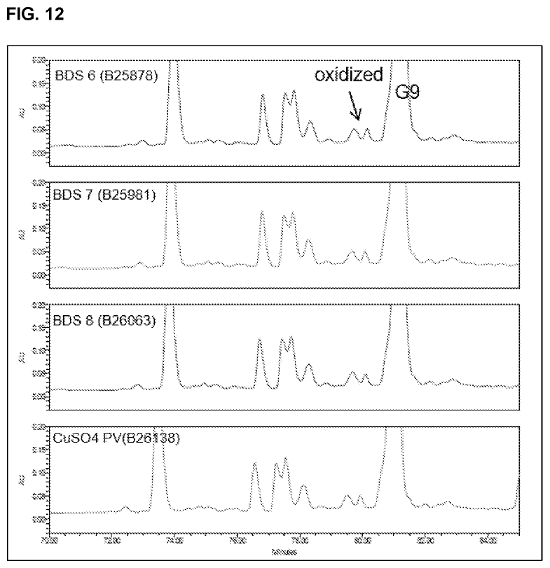

The Glu-C peptide map fragment G9 (residues 61-93) contains two Methionine's (M 79 and M 80). Oxidized methionine at position 79 can be detected on the RP-HPLC chromatogram as it elutes prior to the G9 peak (previously determined by ESI-MS/MS). Methionine 80 is not observed but cannot be completely excluded. The percent oxidation at Methionine 79 show comparability between the CuSO4 batch at BDS (CuSO4 PV) are comparable to results in commercial BDS batches 6-8 batch (CuSO4 PV) and the commercial BDS batches (BDS 6-8). Results are shown in Table 14 and FIG. 12.

TABLE 14
Percent oxidation at Methionine 79

| Test Sample | % Oxidation at Methionine 79 |
|---|---|
| BDS 6 (B25878) | 4.0 |
| BDS 7 (B25981) | 3.8 |
| BDS 8 (B26063) | 3.9 |
| CuSO4 PV (B26138) | 4.0 |

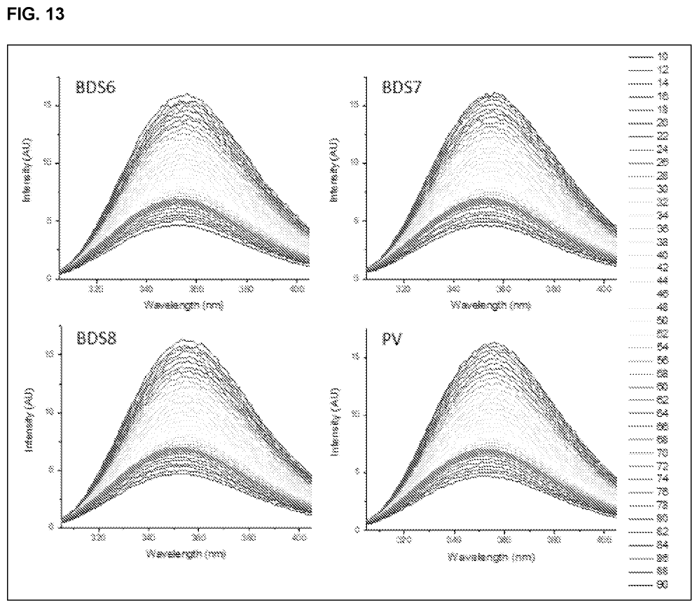

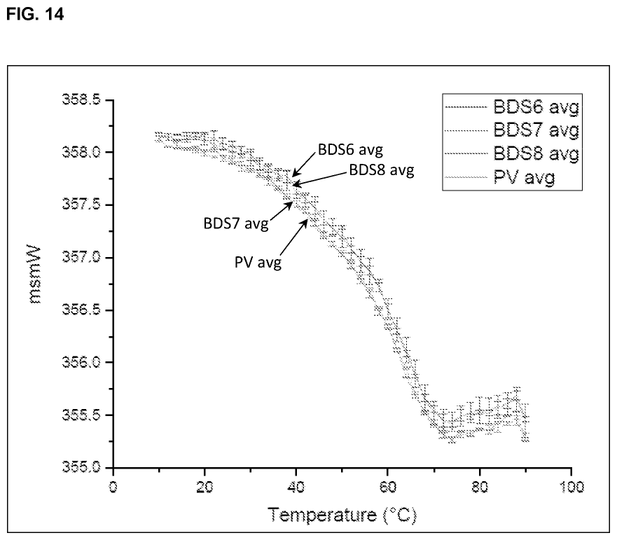

Intrinsic Fluorescence was used to determine the tertiary structure of the proteins by measuring shift in emission maximum wavelength as a function of temperature to monitor the thermal stability of the lots. The fluorescence spectra and thermal unfolding data (Tm) show comparability amongst the four BDS lots tested (CuSO4 PV and the commercial BDS batches (BDS 6-8). Results are shown in FIG. 13 and FIG. 14 and Table 15.

TABLE 15
Tm and Tonset by Spectral Center of Mass of Fluorescence Spectra

| Lot | Tm (° C.) | Tonset (° C.) |
|---|---|---|
| BDS6 | 61.0 + 1.4 | 49.4 + 0.6 |
| BDS7 | 62.0 + 0 | 48.5 + 0.1 |
| BDS8 | 62.0 + 0 | 50.3 + 0.7 |
| PV | 62.0 + 0 | 49.3 + 1.3 |

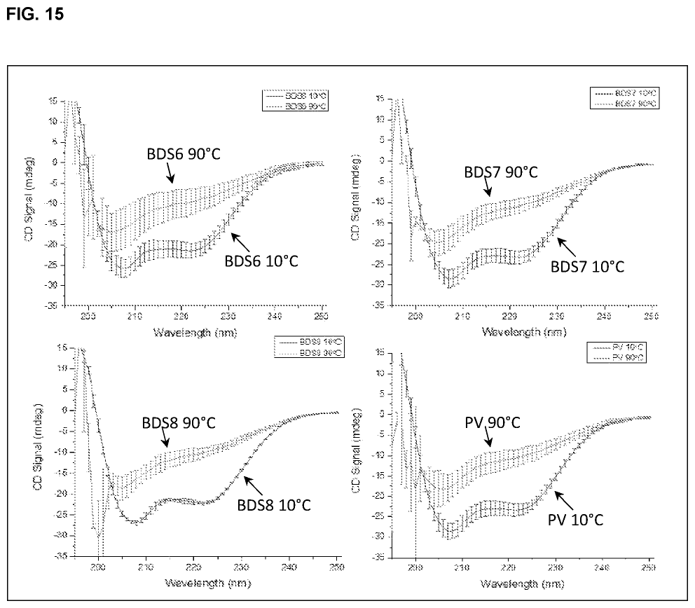

Circular Dichroism (CD) spectroscopy was employed to determine the secondary structure, melting temperature (Tm) and onset of protein unfolding (Tonset) based on the differential absorption of left and right circularly polarized light as a function of temperature. The CD scans with absorbance minima of 208 nm and 222 nm are an indication of predominately alpha helical structures amongst the four BDS lots. The CD scans and thermal unfolding data (Tm and Tonset) show comparability between the CuSO4 batch at BDS (CuSO4 PV) are comparable to results in commercial BDS batches 6-8. Results are shown in FIG. 15 and Table 16, Table 17, and Table 18.

TABLE 16
Tm and Tonset results for 208 nm

| Lot | Tm (° C.) | Tonset (° C.) |
|---|---|---|
| BDS6 | 71.0 + 1.4 | 63.5 + 1.3 |
| BDS7 | 71.0 + 1.4 | 64.7 + 0.1 |
| BDS8 | 71.0 + 1.4 | 64.5 + 0.7 |
| PV | 72.0 + 0 | 63.8 + 1.3 |

TABLE 17
Tm and Tonset results for 218 nm

| Lot | Tm (° C.) | Tonset (° C.) |
|---|---|---|
| BDS6 | 71.0 + 1.4 | 63.2 + 0.4 |
| BDS7 | 71.0 + 1.4 | 63.4 + 0.7 |
| BDS8 | 72.0 + 0 | 63.9 + 0.2 |
| PV | 72.0 + 0 | 64.9 + 0.4 |

TABLE 18
Tm and Tonset results for 222 nm

| Lot | Tm (° C.) | Tonset (° C.) |
|---|---|---|
| BDS6 | 71.0 + 1.4 | 63.4 + 0.3 |
| BDS7 | 71.0 + 1.4 | 63.9 + 1.0 |
| BDS8 | 72.0 + 0 | 64.0 + 0.8 |
| PV | 72.0 + 0 | 64.2 + 0.4 |

Intact mass analysis by MALDI-MS (Matrix Assisted Laser Desorption Ionization Mass Spectrometry) is a method that can provide data on structural integrity and protein modifications by matching the observed spectral masses to theoretical molecular masses based on the amino acid sequence of sargramostim (SEQ ID NO: 2) and associated modifications.

MALDI-MS was done on an Applied Biosystems 4800 MALDI-TOF/TOF. The samples were diluted 10-fold with sinnapinic acid, spotted on a MALDI plate, and MS were acquired for 15 minutes per sample from 2 to 19 KDa.

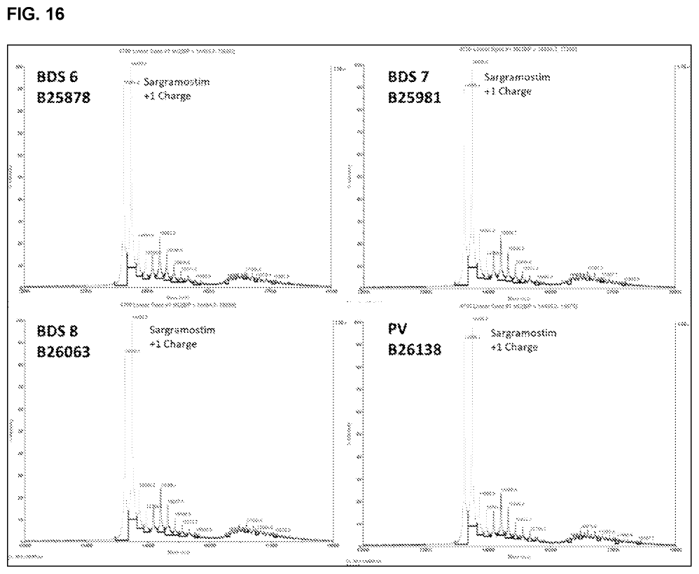

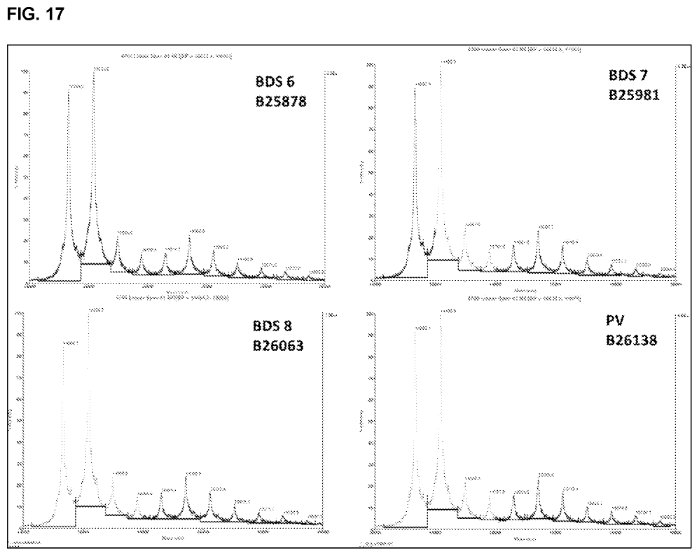

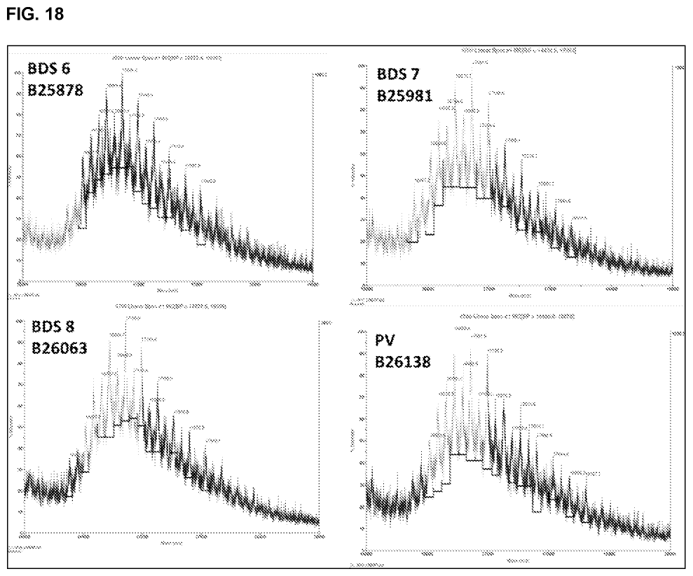

Intact MALDI-MS confirmed sargramostim and glycan molecular weights across lots. FIG. 16 shows the full MALDI mass spectra from 12 to 19 KDa, FIG. 17 shows sargramostim from 14 to 16 KDa, and FIG. 18 shows sargramostim plus glycan from 16 to 19 KDa. The corresponding identifications of the observed mass peaks are given in Table 19.

These results show comparable MALDI-MS profiles and masses, confirming the protein and glycan show comparability between the CuSO4 batch at BDS (CuSO4 PV) are comparable to results in commercial BDS batches 6-8.

TABLE 19
Observed MALDI-MS Masses and Identifications (Putative structure based on
theoretical amino acid and glycan masses)

| | Theoretical | Observed mass (Da) | | | |
|---|---|---|---|---|---|
| Putative Structure* | mass (Da) | BDS 6 | BDS 7 | BDS 8 | PV |
| GM-CSF, -Ala-Pro | 14262 | 14264 | 14266 | 14266 | 14268 |
| GM-CSF, no oligos | 14430 | 14433 | 14435 | 14435 | 14437 |
| GM-CSF, +1 mannose | 14592 | 14595 | 14597 | 14596 | 14599 |
| GM-CSF, +2 mannose | 14755 | 14759 | 14760 | 14760 | 14762 |
| GM-CSF, +3 mannose | 14917 | 14920 | 14922 | 14921 | 14923 |
| GM-CSF, +4 mannose | 15079 | 15084 | 15085 | 15085 | 15087 |
| GM-CSF, +5 mannose | 15241 | 15246 | 15247 | 15247 | 15249 |
| GM-CSF, +6 mannose | 15402 | 15409 | 15410 | 15409 | 15411 |
| GM-CSF, +7 mannose | 15564 | 15571 | 15571 | 15573 | 15575 |
| GM-CSF, +8 mannose | 15726 | 15733 | 15733 | 15735 | 15736 |
| GM-CSF, +9 mannose | 15889 | 15894 | 15896 | 15895 | 15896 |
| GM-CSF, +2 NAcGlucosamine, +10 | 16459 | 16465 | 16467 | 16463 | ND |
| mannose | | | | | |
| GM-CSF, +2 NAcGlucosamine, +11 | 16621 | 16627 | 16626 | 16628 | 16630 |
| mannose | | | | | |
| GM-CSF, +2 NAcGlucosamine, +11 | 16701 | 16706 | 16707 | 16704 | 16711 |
| mannose, +1 phosphate | | | | | |
| GM-CSF, +2 NAcGlucosamine, +12 | 16783 | 16787 | 16789 | 16788 | 16792 |
| mannose | | | | | |
| GM-CSF, +2 NAcGlucosamine, +12 | 16863 | 16868 | 16871 | 16869 | 16871 |
| mannose, +1 phosphate | | | | | |
| GM-CSF, +2 NAcGlucosamine, +13 | 16946 | 16948 | 16948 | 16946 | 16953 |
| mannose | | | | | |
| GM-CSF, +2 NAcGlucosamine, +13 | 17026 | 17029 | 17031 | 17030 | 17032 |
| mannose, +1 phosphate | | | | | |
| GM-CSF, +2 NAcGlucosamine, +14 | 17108 | 17104 | 17109 | 17106 | 17109 |
| mannose | | | | | |
| GM-CSF, +2 NAcGlucosamine, +14 | 17188 | 17192 | 17193 | 17193 | 17196 |
| mannose, +1 phosphate | | | | | |
| GM-CSF, +2 NAcGlucosamine, +15 | 17270 | 17270 | 17277 | 17270 | 17275 |
| mannose | | | | | |
| GM-CSF, +2 NAcGlucosamine, +15 | 17350 | 17353 | 17355 | 17355 | 17348 |
| mannose, +1 phosphate | | | | | |
| GM-CSF, +2 NAcGlucosamine, +16 | 17432 | 17428 | 17437 | 17430 | 17439 |
| mannose | | | | | |
| GM-CSF, +2 NAcGlucosamine, +16 | 17512 | 17515 | 17519 | 17517 | 17521 |
| mannose, +1 phosphate | | | | | |
| GM-CSF, +2 NAcGlucosamine, +17 | 17594 | 17608 | 17603 | 17592 | 17603 |
| mannose | | | | | |
| GM-CSF, +2 NAcGlucosamine, +17 | 17674 | 17682 | 17680 | 17678 | 17683 |
| mannose, +1 phosphate | | | | | |
| GM-CSF, +2 NAcGlucosamine, +18 | 17836 | 17843 | 17844 | 17842 | 17845 |
| mannose, +1 phosphate | | | | | |

Host Cell Protein (HCP) analysis by Proteomic LC-MS/MS is a method for globally identifying and quantitating low abundance proteins in a sample. To identify HCPs in commercial lots BDS 6-8, and CuSO4 supplemented PV lot (PV), the BDS was proteolyzed with trypsin, and separated by reversed phase C18 nano-LC over a 60 minute gradient. Tandem mass spectra of the LC peaks were generated on an Orbitrap Elite ETD mass spectrometer, and protein identities were detected using the Protein Metrics database and spectral analysis software. Relative quantities of the yeast HCPs in each sample were generated from the extracted ion signal (XIC) for each peptide and compared across lots for this analysis.

The identified proteins at 0.01% XIC area are described in Table 20. For the % XIC values, the upper number in each cell describes the value relative to all identified proteins. The lower number in parenthesis describes the relative value when the method artifact contaminants are removed.

The results show that all the lots contain at least 99% sargramostim by ion signal, indicating most HCPs are removed during the purification steps. In addition, the low abundance HCPs that were identified are comparable across lots. Thus, the identified HCP profile in the CuSO4 supplemented PV Lot at BDS (CuSO4 PV) is comparable to results in commercial BDS batches 6-8.

TABLE 20
Proteins with >0.01% XIC Signal in Proteomic LC-MS/MS Analysis for HCPs

| | | | Relative % XIC Signal of All IDs and | | | | |
|---|---|---|---|---|---|---|---|
| | | | (% XIC without contaminants) | | | | |
| | | | BDS6 | BDS7 | BDS8 | PV | |
| Protein ID | Organism | Description | B256878 | B25981 | B26063 | B26138 | Source |
| Sargram | Human | Sargramostim | 97.35 | 97.41 | 98.01 | 97.95 | Drug |
| | | | (99.91) | (99.93) | (99.88) | (99.81) | Substance |
| GP179 | Human | Probable G- | 1.84 | 2.50 | 1.83 | 1.82 | Method |
| | | Protein Coupled | (NA | (NA) | (NA) | (NA) | contaminant |
| | | Receptor 179 | | | | | |
| K1C9 | Human | Keratin Type 1 | 0.70 | 0.00 | 0.02 | 0.02 | Method |
| | | | (NA) | (NA) | (NA) | (NA) | contaminant |
| SODM | Yeast | Superoxide | 0.00 | 0.01 | 0.02 | 0.03 | HCP |
| | | dismutase | (0.01) | (0.01) | (0.02) | (0.03) | |
| CYPB | Yeast | Peptidyl-prolyl | 0.02 | 0.01 | 0.03 | 0.01 | HCP |
| | | cis-trans | (0.02) | (0.01) | (0.03) | (0.01) | |
| | | isomerase B | | | | | |
| CYPH | Yeast | Peptidyl-prolyl | 0.01 | 0.01 | 0.02 | 0.02 | HCP |
| | | cis-trans | (0.01) | (0.01) | (0.02) | (0.03) | |
| | | isomerase B | | | | | |
| YHT8 | Yeast | Uncharacterized | 0.00 | 0.01 | 0.02 | 0.02 | HCP |
| | | protein YHR138C | (0.00) | (0.01) | (0.02) | (0.02) | |
| 6P22 | Yeast | 6-phosphofructo- | 0.02 | 0.00 | 0.00 | 0.01 | HCP |
| | | 2-kinase 2 | (0.02) | (0.00) | (0.00) | (0.01) | |
| GPX3 | Yeast | Glutathione | 0.00 | 0.00 | 0.01 | 0.03 | HCP |
| | | peroxidase-like | (0.00) | (0.00) | (0.01) | (0.03) | |
| | | peroxiredoxin | | | | | |
| | | HYR1 | | | | | |
| EF3A | Yeast | Elongation factor | 0.00 | 0.01 | 0.00 | 0.01 | HCP |
| | | 3A | (0.01) | (0.01) | (0.00) | (0.01) | |
| FKBP2 | Yeast | Peptidyl-prolyl | 0.01 | 0.00 | 0.01 | 0.01 | HCP |
| | | cis-trans | (0.01) | (0.00) | (0.01) | (0.01) | |
| | | isomerase FPR2 | | | | | |
| CSF2 | Human | GM-CSF | 0.00 | 0.00 | 0.00 | 0.01 | Drug |
| | | | (0.00) | (0.00) | (0.00) | (0.01) | Substance |
| CYPD | Yeast | Peptidyl-prolyl | 0.00 | 0.00 | 0.00 | 0.01 | HCP |
| | | cis-trans | (0.00) | (0.00) | (0.00) | (0.01) | |
| | | isomerase D | | | | | |
| SGT2 | Yeast | Small glutamine- | 0.00 | 0.00 | 0.00 | 0.01 | HCP |
| | | rich | (0.00) | (0.00) | (0.00) | (0.01) | |
| | | tetratricopeptide | | | | | |
| | | repeat-containing | | | | | |
| | | protein | | | | | |
| HMF1 | Yeast | HMF1 | 0.01 | 0.00 | 0.01 | 0.00 | HCP |
| | | | (0.01) | (0.00) | (0.01) | (0.00) | |
| PDI | Yeast | Disulfide- | 0.00 | 0.00 | 0.00 | 0.01 | HCP |
| | | isomerase | (0.00) | (0.00) | (0.00) | (0.01) | |
| TEN2 | Human | Tenurin-2 | 0.00 | 0.00 | 0.00 | 0.01 | Method |
| | | | (NA) | (NA) | (NA) | (NA) | Contaminant |

Testing for elemental impurities (including copper) was performed on two commercial BDS batches (BDS 7 and BDS 8) and on the one CUSO4 supplemented process validation BDS batch (BDS PV) via ICP-MS Quantitative Screen Test Elements selected for testing follow the recommendations of ICH Q3D (R1), Guideline for Elemental Impurities (22 Mar. 2019). Additionally, molybdenum was included in the testing plan because it is intentionally added to the process in trace amounts. Results (Table 21) demonstrated that the impurity profile of the process validation batch was consistent with recent commercial batches and are reported as less than the limit of quantitation (LOQ) for the assay. Although Cu was introduced during the upstream cell culture processing, the data demonstrated that the elemental impurities, including Cu, were subsequently reduced during the downstream processing (as expected). All results were below the Maximum Permissible Concentration (MPC) and Control Threshold (CT) limits. All results correspond to less than the Permitted Daily Exposure for a parenteral drug product as described in ICH Q3D.

TABLE 21
Elemental Impurities (ppb)

| | Elemental Impurities (ppb) | | | | | | Below |
|---|---|---|---|---|---|---|---|
| | BDS 7 | BDS 8 | PV | | | | Exposure |
| Element | B25981 | B26063 | B26138 | MPC | CT | Comparable | Limits |
| Lithium | <10 | <10 | <10 | 5.00E+08 | 1.50E+08 | Yes | Yes |
| Vanadium | <10 | <10 | <10 | 2.00E+07 | 6.00E+06 | Yes | Yes |
| Cobalt | <10 | <10 | <10 | 1.00E+07 | 3.00E+06 | Yes | Yes |
| Nickel | 10 | <10 | <10 | 4.00E+07 | 1.20E+07 | Yes | Yes |
| Copper | <10 | <10 | <10 | 6.00E+08 | 1.80E+08 | Yes | Yes |
| Arsenic | <10 | <10 | <10 | 3.00E+07 | 9.00E+06 | Yes | Yes |
| Molybdenum | <10 | <10 | <10 | 3.00E+09 | 9.00E+08 | Yes | Yes |
| Cadmium | <10 | <10 | <10 | 4.00E+06 | 1.20E+06 | Yes | Yes |
| Antimony | <10 | <10 | <10 | 1.80E+08 | 5.40E+07 | Yes | Yes |
| Mercury | <10 | <10 | <10 | 6.00E+06 | 1.80E+06 | Yes | Yes |
| Lead | <10 | <10 | <10 | 1.00E+07 | 3.00E+06 | Yes | Yes |

Threshold DNA testing was performed to verify that residual DNA levels in the BDS are cleared given the increased biomass from the CuSO4 process. Acceptance criteria was based on the historical BDS specification (Note: Residual DNA was removed as a product release criteria per change control MOC-00074 in August 2020.) The Process Validation BDS CuSO4 batch at BDS (PV) result met the acceptance criteria, refer to Table 22 below:

| | Acceptance | |
|---|---|---|
| Test/Method | Criteria | PV B26138 Result |
| Threshold DNA/T-0401 | ≤10 pg/mg | -0.2 pg/mg* |

*To eliminate slight positive bias in mean quantitation of samples with no DNA, the
standard curve for the Threshold DNA Assay uses an extended power fit algorithm which
forces the regression line through zero; this enables quantitation of near zero negative
values.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

As used herein, all headings are simply for organization and are not intended to limit the disclosure in any manner. The content of any individual section may be equally applicable to all sections.

What is claimed is:

1. A method for production of a recombinant protein, comprising
(a) adding a trace element to a culture medium comprising a host cell, the host cell comprising a nucleic acid molecule encoding the recombinant protein and being capable of producing the recombinant protein during fermentation, and
(b) isolating the recombinant protein,
wherein the trace element is exogenously added to the culture medium to supplement an amount of trace element in the culture medium.

2. The method of claim 1, wherein the recombinant protein is recombinant human granulocyte macrophage-colony stimulating factor (rhu GM-CSF) protein, comprising an amino acid sequence having at least about 97% identity with SEQ ID NO: 1 or SEQ ID NO: 2.

3. The method of claim 1 or 2, wherein the recombinant protein binds and/or activates the granulocyte-macrophage colony stimulating factor receptor (GM-CSF-R-alpha or CSF2R).

4. The method of claim 1, wherein the addition of the trace element during production of the recombinant protein increases expression levels of the recombinant protein, as compared to a method without the addition of the trace element.

5. The method of claim 1, wherein the addition of the trace element during the production of the recombinant protein improves the fermentation yield of said recombinant protein, as compared to a method without the addition of the trace element.

6. The method of claim 1, wherein the addition of the trace element improves the consistency of the fermentation performance during the production of the recombinant protein, as compared to a method without the addition of the trace element.

7. The method of claim 1, wherein the trace element is copper.

8. The method of claim 7, wherein the copper is in the form of a copper derivative.

9. The method of claim 7, wherein the copper is in the form of a copper compound.

10. The method of claim 8 or 9, wherein the copper is a copper salt.

11. The method of claim 10, wherein the copper salt is cupric or copper sulfate.

12. The method of any one of claims 7-11, wherein copper is added to the culture medium in an amount of about 0.5 μM to about 100 μM, optionally being about 0.5 μM to about 80 μM, or optionally being about 1 μM to about 20 μM.

13. The method of any one of claims 1-12, wherein the nucleic acid molecule is a vector.

14. The method of claim 13, wherein the nucleic acid molecule has a codon-optimized sequence.

15. The method of any one of claims 1-14, wherein the host cell expresses the recombinant protein.

16. The method of claim 15, wherein the host cell is a non-human host cell.

17. The method of claim 16, wherein the non-human host cell is a yeast cell or mammalian cell, optionally being a Chinese hamster ovary (CHO) cell.

18. The method of claim 17, wherein the yeast cell is a non-methylotrophic yeast cell.

19. The method of claim 18, wherein the host cell is a Saccharomyces cerevisiae cell.

20. A pharmaceutical composition comprising a recombinant human GM-CSF obtained using the method of any one of claims 1-19 and a pharmaceutically acceptable excipient or carrier.

21. A method of treating a patient or subject who is undertaking or has undertaken a cancer therapy, or who is undertaking, or has undertaken a therapy against an infectious agent and/or has undertaken a therapy to treat the effects of an infectious disease, or who is undertaking or has undertaken a bone marrow transplant, and/or who had been acutely exposed to myelosuppressive doses of radiation; the method comprising administering to the patient a therapeutically effective amount of the pharmaceutical composition of claim 20.

22. The method of claim 21, wherein the patient is treated by modulating clonal expansion, survival, differentiation and activation state of hematopoietic progenitor cells.

23. The method of claim 21, wherein the patient is treated by modulating a myelomonocytic cell lineage, by promoting the proliferation of megakaryocytic and erythroid progenitors.

24. The method of claim 21, wherein the patient is treated by modulating hematopoietic progenitor cells, by stimulating the survival, proliferation and activation of neutrophils, macrophages and/or dendritic cells.

25. The method of claim 21, wherein the patient is treated following bone marrow transplant by modulating hematopoietic progenitor cells, by stimulating the survival, proliferation and activation of neutrophils, macrophages and/or dendritic cells.

26. A method of therapy, comprising administering to a patient a therapeutically effective amount of the pharmaceutical composition of claim 20 or contacting cells with an effective amount of the pharmaceutical composition of claim 20 and administering therapeutically effective amount of the cells, wherein the therapy:
accelerates neutrophil recovery and/or to reduce the incidence of infections following induction chemotherapy;
mobilizes hematopoietic progenitor cells into peripheral blood for collection by leukapheresis and transplantation;
accelerates of myeloid reconstitution following autologous or allogeneic bone marrow or peripheral blood progenitor cell transplantation;
treats delayed neutrophil recovery or graft failure after autologous or allogeneic bone marrow transplantation;
treats hematopoietic syndrome of acute radiation syndrome (H-ARS); and/or
treats the sequelae and long-term effects of an infectious disease.

27. A method for treating an infection with a virus, comprising: administering an effective amount of a composition comprising the pharmaceutical composition of claim 20 a patient in need thereof.

28. The method of claim 27, wherein the virus is an influenza or a coronavirus, the coronavirus optionally being a betacoronavirus, optionally selected from severe acute respiratory syndrome coronavirus 2 (SARS-CoV-2), SARS-CoV, Middle East respiratory syndrome-corona virus (MERS-CoV), HCoV-HKU1, and HCoV-OC43 or an alphacoronavirus, optionally selected from HCoV-NL63 and HCoV-229E.

29. The method of claim 28, wherein the coronavirus is SARS-CoV-2.

30. The method of claim 29, wherein the patient is afflicted with COVID-19.

31. The method of any one of claims 26-30, wherein the patient is afflicted with one or more of fever, cough, shortness of breath, diarrhea, upper respiratory symptoms, lower respiratory symptoms, pneumonia, and acute respiratory syndrome.

32. The method of any one of claims 26-31, wherein the patient is hypoxic.

33. The method of any one of claims 26-32, wherein the patient is afflicted with respiratory distress.

34. The method of any one of claims 26-33, wherein the method prevents or mitigates development of acute respiratory distress syndrome (ARDS) in the patient.

35. The method of any one of claims 26-34, wherein the method improves oxygenation in the patient.

36. The method of any one of claims 26-35, wherein the method prevents or mitigates a transition from respiratory distress to cytokine imbalance in the patient.

37. The method of any one of claims 26-36, wherein the method reverses or prevents a cytokine storm.

38. The method of claim 37, wherein the method reverses or prevents a cytokine storm in the lungs or systemically.

39. The method of claim 37 or 38, wherein the cytokine storm is selected from one or more of systemic inflammatory response syndrome, cytokine release syndrome, macrophage activation syndrome, and hemophagocytic lymphohistiocytosis.

40. The method of claim 37 or 38, wherein the method reverses or prevents excessive production of one or more inflammatory cytokines.

41. The method of claim 40, wherein the inflammatory cytokine is one or more of IL-6, IL-1, IL-1 receptor antagonist (IL-1ra), IL-2ra, IL-10, IL-18, TNFα, interferon-γ, CXCL10, and CCL7.

42. The method of any one of claims 26-41, wherein the method causes a decrease in viral load in the patient relative to before treatment.

43. A method for treating or preventing a viral infection in a subject in need thereof, comprising:
providing plasma from a donor subject who has recovered from the viral infection,
the plasma comprising IgG, IgM and/or IgA antibodies directed against the virus causing the infection and
the donor subject having been treated with the pharmaceutical composition of claim 20 to stimulate production of the antibodies; and
administering the plasma to the subject in need thereof.

44. A method for treating or preventing a viral infection in a subject in need thereof, comprising:
administering the pharmaceutical composition of claim 20 to a donor subject who has recovered from the viral infection;
isolating plasma from the donor subject, the plasma comprising IgG, IgM and/or IgA antibodies directed against the virus causing the infection; and
administering the plasma to the subject in need thereof.

45. The method of claim 43 or 44, wherein the method provides passive immunization against the virus to the subject in need thereof.

46. The method of any one of claims 43-45, wherein the IgG, IgM and/or IgA antibodies specifically bind to a viral antigen.

47. The method of claim 46, wherein the IgG, IgM and/or IgA antibodies neutralize the virus.

48. The method of claim 46 or 47, wherein the IgG, IgM and/or IgA antibodies prevent or diminish infection of a cell by the virus.

49. The method of any one of claims 43-48, wherein the viral infection is selected from a betacoronavirus infection, optionally selected from severe acute respiratory syndrome coronavirus 2 (SARS-CoV-2), severe acute respiratory syndrome coronavirus (SARS-CoV-1), Middle East Respiratory Syndrome-Corona Virus (MERS-CoV), HCoV-HKU1, and HCoV-OC43 infection.

50. The method of any one of claims 43-49, wherein the viral infection is selected from an alphacoronavirus infection, optionally selected from HCoV-NL63 and HCoV-229E infection.

51. The method of claim 50, wherein the betacoronavirus infection is severe acute respiratory syndrome (SARS).

52. The method of claim 50, wherein the betacoronavirus infection is, or is associated with, coronavirus disease 2019 (COVID-19).

53. The method of any one of claims 43-52, wherein the viral infection is an influenza infection, optionally selected from Type A, Type B, Type C, and Type D influenza virus infection.

54. The method of claim 53, wherein the influenza infection is pandemic 2009 influenza A (H1N1) or avian influenza A (H5N1).

55. The method of any one of claims 43-54, wherein the donor subject has tested positive for the viral infection prior to recovery.

56. The method of any one of claims 43-55, wherein the donor subject has resolution of viral infection symptoms prior to donation.

57. The method of any one of claims 43-56, wherein the donor subject has tested positive for antibodies directed against the virus using a serological test.

58. The method of any one of claims 43-57, wherein the donor subject demonstrates measurable neutralizing antibody titers.

59. The method of claim 58, wherein the neutralizing antibody titers are at least about 1:160.

60. The method of any one of claims 43-59, wherein the plasma is isolated from a blood sample from the donor subject.

61. The method of claim 60, wherein the plasma is isolated via plasmapheresis.

62. The method of any one of claims 43-61, wherein the plasma comprises a therapeutically effective amount of the IgG, IgM and/or IgA antibodies directed against the virus causing the infection.

63. A method for production of a recombinant protein, comprising
(a) adding a copper salt to a culture medium comprising a host cell, the host cell comprising a nucleic acid molecule encoding the recombinant protein and being capable of producing the recombinant protein during fermentation, and
(b) isolating the recombinant protein, wherein:
the copper salt is exogenously added in amount of about 1 μM to about 20 μM to the culture medium to supplement an amount of trace element in the culture medium;
the copper salt is cupric or copper sulfate; and
the recombinant protein is recombinant human granulocyte macrophage-colony stimulating factor (rhu GM-CSF) protein having at least about 97% identity with SEQ ID NO: 2.

64. The method of claim 63, wherein the addition of the trace element during production of the recombinant protein increases expression levels of the recombinant protein, as compared to a method without the addition of the trace element.


SEQUENCE LISTING (2 sequences)

SEQ ID NO: 1 - protein, 127 aa, Artificial Sequence [sequence omitted]

SEQ ID NO: 2 - protein, 127 aa, Artificial Sequence
  Synthetic Sequence
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE